(19) United States
(12) Patent Application Publication
(10) Pub. No.: US 2025/0210142 A1
(43) Pub. Date: Jun. 26, 2025

(54) Method For Managing Multiple Parallel Library Preparation Workflows With Shared Resources

(71) Applicant: ILLUMINA, INC., San Diego, CA (US)

(72) Inventors: Gregory Holst, San Diego, CA (US); Emily Parker, Encinitas, CA (US); Bryan Crane, San Diego, CA (US); Peter Thomas, Poway, CA (US)

(21) Appl. No.: 18/979,039

(22) Filed: Dec. 12, 2024

Related U.S. Application Data
(60) Provisional application No. 63/613,180, filed on Dec. 21, 2023.

(51) Int. Cl. G16B 35/00; G06F 9/48

(52) U.S. Cl. CPC G16B 35/00; G06F 9/4843

(57) ABSTRACT

A system for managing multiple parallel library preparation workflows with shared resources includes at least two assay bays configured to prepare a library of samples according to a workflow. A common resource is configured to assist in preparation of the library of samples according to a common resource schedule. The system also includes a scheduler for scheduling workflows according to the common resource schedule. The system receives a first workflow scheduling request for a first assay bay that utilizes the common resource, and a second workflow scheduling request for a second assay bay that utilizes the common resource. The system determines that the first and second workflows conflict in time or space with respect to the common resource, obtains characteristics of the first and second workflows, and assigns the first and second workflows to the common resource schedule based on the characteristics to resolve the conflict.

### CROSS-REFERENCE TO RELATED APPLICATIONS

This application claims priority to U.S. Provisional Patent Application No. 63/613,180, filed Dec. 21, 2023, entitled “Method for Managing Multiple Parallel Library Preparation Workflows with Shared Resources,” the entire disclosure of which is hereby expressly incorporated by reference herein.

### FIELD OF DISCLOSURE

The present application generally relates to library preparation workflows, and more specifically to managing multiple parallel library preparation workflows with shared resources.

### BACKGROUND

DNA libraries may be prepared using an associated workflow. In systems with multiple components to prepare a library, such as an assay bay and common resource, each workflow may be highly complex and compete with other workflows for the limited resources of a library preparation system, constraining the workflows and reducing associated run times. Workflow management needs to be generalized across multiple workflow types with various levels of the shared resource usage to guarantee the requirements of the library preparation.

### SUMMARY

Shortcomings of the prior art can be overcome, and benefits as described in this disclosure can be achieved, through systems and methods for managing multiple parallel library preparation workflows with shared resources. Various implementations of the systems and methods are described below, and the systems and methods in any combination (provided these combinations are not inconsistent), may overcome these shortcomings and achieve the benefits described herein.

In a library preparation system, samples are prepared using workflows. The samples may be prepared separately in different assay bays each having their own workflow before they are pooled together in a library of samples. The library preparation system may also include a common resource across the different assay bays which may be used to assist in preparing the samples in each of the assay bays. For example, the common resource may include a movable common resource component such as a mover operatively coupled to the common resource. The movable common resource component may move in a first direction parallel to the assays bays and a second direction perpendicular to the first direction (e.g., across the assay bays) via a gantry system.

In some scenarios, multiple workflows in different assay bays may require the use of the common resource at the same time. Additionally in some scenarios, the common resource may collide with components within each assay bay when attempting to complete the workflows, such as a movable assay bay component (e.g., a contact dispenser) which is linearly movable in a first direction parallel to the assay bay.

To address these conflicts in time or in space, the library preparation system may include a scheduler that assigns workflows to a common resource schedule. The workflows may be assigned to the common resource schedule using various methods, which include:
1) First come, first serve/wait until available,
2) Prioritization,
3) Resource stealing,
4) Resource Future Reservation,
5) Spatial Resource Discretization,
6) Spatial Resource Collision Detection,
7) Pre-allocated Resource Time, and/or
8) Parallel Operation Start/End Flexibility.

Then the scheduler may send control signals to the common resource, the components within the assay bays, and/or other components of the library preparation system to cause the components to carry out the workflows in accordance with the common resource schedule.

For example, the scheduler may receive a first workflow request for a first assay bay, and a second workflow request for a second assay bay, and determine that the first and second workflows conflict in time or space with respect to a common resource. Then the scheduler may assign the first and second workflows to the common resource schedule based upon one or more characteristics of each workflow, e.g., temporal characteristics (run time, wait time, priority, etc.), and/or spatial characteristics (e.g., location of objects/components). Based upon the workflow characteristics, the scheduler assigns the workflows in a manner which resolves the conflict with respect to the common resource.

In a first implementation, a system for managing multiple parallel library preparation workflows with shared resources includes one or more processors; a non-transitory computer-readable memory; at least two assay bays, each assay bay configured to prepare a library of samples according to a workflow including workflow characteristics; a common resource configured to assist in preparation of the library of samples according to a common resource schedule; and a scheduler stored on the memory for scheduling workflows according to the common resource schedule that, when executed by the one or more processors. The scheduler may cause the system to: (i) receive, for a first assay bay of the at least two assay bays, a first workflow scheduling request including a first workflow that utilizes the common resource; (ii) receive, for a second assay bay of the at least two assay bays, a second workflow scheduling request including a second workflow that utilizes the common resource; (iii) determine that the first and second workflows conflict in time or space with respect to the common resource; (iv) obtain one or more first characteristics of the first workflow and one or more second characteristics of the second workflow; and (v) assign the first and second workflows to the common resource schedule based on the one or more first characteristics and the one or more second characteristics, wherein the common resource schedule resolves the conflict between the first and second workflows with respect to the common resource.

In a second implementation, a computer-implemented method is utilized for managing multiple parallel library preparation workflows with shared resources. The method may include (i) receiving, by one or more processors, for a first assay bay of at least two assay bays, a first workflow scheduling request including a first workflow that utilizes a common resource, wherein (a) each assay bay of the at least two assay bays is configured to prepare a library of samples according to a workflow including workflow characteristics; and (b) the common resource is configured to assist in preparation of a library of samples according to a common resource schedule; (ii) receiving, by one or more processors, for a second assay bay of the at least two assay bays, a second workflow scheduling request including a second workflow that utilizes the common resource; (iii) determining, by the one or more processors, that the first and second workflows conflict in time or space with respect to the common resource; (iv) obtaining, by the one or more processors, one or more first characteristics of the first workflow and one or more second characteristics of the second workflow; and (v) assigning, by the one or more processors, the first and second workflows to the common resource schedule based on the one or more first characteristics and the one or more second characteristics, wherein the common resource schedule resolves the conflict between the first and second workflows with respect to the common resource.

In a third implementation, a non-transitory computer-readable medium stores processor-executable instructions that, when executed by one or more processors, may cause the one or more processors to: (i) receive, for a first assay bay of at least two assay bays, a first workflow scheduling request including a first workflow that utilizes a common resource, wherein (a) each assay bay of the at least two assay bays is configured to prepare a library of samples according to a workflow including workflow characteristics; and (b) the common resource is configured to assist in preparation of a library of samples according to a common resource schedule; (ii) receive, for a second assay bay of the at least two assay bays, a second workflow scheduling request including a second workflow that utilizes the common resource; (iii) determine that the first and second workflows conflict in time or space with respect to the common resource; (iv) obtain one or more first characteristics of the first workflow and one or more second characteristics of the second workflow; and (v) assign the first and second workflows to the common resource schedule based on the one or more first characteristics and the one or more second characteristics, wherein the common resource schedule resolves the conflict between the first and second workflows with respect to the common resource.

In further accordance with the foregoing the first, second, and/or third implementations, the system, method apparatus and/or non-transitory computer-readable medium may further include or comprise any one or more of the following features.

In an implementation, the one or more first and second characteristics include temporal characteristics including one or more of: a maximum wait time, a minimum wait time, a maximum run time, a minimum run time, a proposed scheduled time, or a priority level.

In another implementation, a first portion of the first workflow and a second portion of the second workflow that utilize the common resource are assigned to the common resource schedule in an order in which the respective first and second workflow requests are received by the scheduler.

In another implementation, assigning the first and second portions of the first and second workflow to the common resource schedule in the order in which the respective first and second workflow requests are received includes determining that the first workflow request is received before the second workflow request, and adding a wait time to a requested time of the second portion until the common resource becomes available.

In another implementation, the one or more first characteristics include a first priority level for the first workflow. The one or more second characteristics include a second priority level for the second workflow. Assigning the first and second workflows to the common resource schedule includes assigning a first portion of the first workflow and a second portion of the second workflow that requires use of the common resource to the common resource schedule in an order according to the first and second priority levels of the first and second workflows. A higher priority workflow is scheduled before a lower priority workflow.

In another implementation, the method further comprises assigning a second portion of the second workflow that utilizes the common resource to the common resource schedule at an intermediate time between a start of a first portion of the first workflow that utilizes the common resource and an end of the first portion.

In another implementation, the method further comprises assigning a second portion of the second workflow that utilizes the common resource to the common resource schedule at an intermediate time between a start of a first portion of the first workflow that utilizes the common resource and an end of the first portion.

In another implementation, the one or more first characteristics include a maximum wait time of the first workflow. Assigning the second portion to the common resource schedule at the intermediate time between the start and end of the first portion includes verifying that a duration of the second portion does not exceed the maximum wait time of the first workflow.

In another implementation, the method further comprises receiving a third workflow scheduling request for a future workflow that utilizes the common resource. The third workflow scheduling request includes a proposed time for the future workflow. The method also includes determining that the common resource is available at the proposed time, and assigning the future workflow to the common resource schedule at the proposed time.

In another implementation, the method further comprises identifying a spatial conflict between a movable common resource component of the common resource and a movable assay bay component of one of the each assay bays based upon the common resource schedule. Based upon the spatial conflict, the method includes performing one or more of: (i) pausing the first or second workflow associated with the conflict, (ii) adding a wait time to the first or second workflow associated with the conflict, or (iii) causing the movable common resource component or the movable assay bay component to move in a manner which resolves the spatial conflict.

In another implementation, the method further comprises identifying spatial zones representing spaces in which the movable common resource component or the movable assay bay component moves during the first or second workflow. The spatial conflict between the movable common resource component and the movable assay bay component is identified based upon at least one of: (i) a plurality of tasks within the first or second workflows occurring in a same spatial zone, or (ii) a potential collision according to trajectories of the movable common resource component and the movable assay bay component to particular spatial zones.

In another implementation, the movable assay bay component is a contact dispenser controllable via the one or more processors and linearly movable in a first direction parallel to one of the assay bays. The movable common resource component is a mover controllable via the one or more processors and is operatively coupled to the common resource and the assay bays. The mover is movable in the first direction and a second direction perpendicular to the first direction.

In another implementation, the mover is movable along a gantry system.

In another implementation, the one or more first and second characteristics include spatial characteristics and include a location of one or more of: (i) a target object, (ii) the movable assay bay component, or (iii) the movable common resource component.

In another implementation, one or more of the movable common resource component or the movable assay bay component executes at least a portion of the first workflow or the second workflow according to the common resource schedule.

In another implementation, the one or more first characteristics include information identifying an assay bay that initiated the first workflow scheduling request. The method further comprises pre-allocating blocks of time on the common resource schedule to each of the assay bays for utilizing the common resource. Additionally, the method includes assigning a first portion of the first workflow that utilizes the common resource to the common resource schedule at a next pre-allocated block of time for the assay bay that initiated the first workflow scheduling request based upon receiving the first workflow request.

In another implementation, the method further includes determining that at least two operations of the first or second workflows on the common resource schedule can be conducted in parallel, and assigning the at least two operations of the first or second workflows to the common resource schedule to be executed in parallel.

It should be appreciated that all combinations of the foregoing concepts and additional concepts discussed in greater detail below (provided such concepts are not mutually inconsistent) are contemplated as being part of the subject matter disclosed herein and/or may be combined to achieve the particular benefits of a particular aspect described herein. In particular, all combinations of claimed subject matter appearing at the end of this disclosure are contemplated as being part of the subject matter disclosed herein.

### BRIEF DESCRIPTION OF THE DRAWINGS

FIG. 1 illustrates a schematic diagram of an implementation of a system for managing multiple parallel library preparation workflows with shared resources in accordance with the teachings of this disclosure.

FIG. 2 is a top view of a system that can be used to implement the system 100 of FIG. 1.

FIG. 3 is a front view of the system of FIG. 2.

FIG. 4 is an isometric view of the system of FIG. 2.

FIG. 5 is an example block diagram illustrating scheduled workflows for a first assay bay and a second assay.

FIG. 6A is an example block diagram illustrating scheduled workflows for a first assay bay and a second assay bay which include a conflict.

FIG. 6B is an example block diagram illustrating scheduled workflows for a first assay bay and a second assay bay which resolve a conflict using a wait time rule.

FIG. 6C is an example block diagram illustrating scheduled workflows for a first assay bay and a second assay bay which resolve a conflict using a resource stealing rule.

FIG. 6D is an example block diagram illustrating scheduled workflows for a

first assay bay and a second assay bay which resolve a conflict using a future scheduling open time rule.

FIG. 7 is an example block diagram illustrating a pre-allocated workflow schedule.

FIG. 8A is an example block diagram illustrating spatial zones of a system for managing multiple parallel library preparation workflows.

FIG. 8B is an example block diagram illustrating scheduled parallel workflows.

FIG. 9 is a flow diagram of an example computer-implemented method for managing multiple parallel library preparation workflows with shared resources.

### DETAILED DESCRIPTION

Although the following text discloses a detailed description of implementations of methods, apparatuses and/or articles of manufacture, it should be understood that the legal scope of the property right is defined by the words of the claims set forth at the end of this patent. Accordingly, the following detailed description is to be construed as examples only and does not describe every possible implementation, as describing every possible implementation would be impractical, if not impossible. Numerous alternative implementations could be implemented, using either current technology or technology developed after the filing date of this patent. It is envisioned that such alternative implementations would still fall within the scope of the claims.

At least one aspect of this disclosure is related to systems for managing multiple parallel library preparation workflows with shared resources, thereby optimizing and/or improving the utilization of instrument hardware and the overall system operations. The systems disclosed may use at least two assay bays, each configured to prepare a library of samples according to a workflow. The systems may use a common resource (e.g., a common bay) configured to assist in preparation of the library of samples according to a common resource schedule. The systems may use a scheduler for scheduling workflows according to the common resource schedule.

The disclosed implementations enable workflows of library preparation and/or automating assays of varying complexity. The disclosed implementations enable the ability to run multiple assay workflows in parallel and/or the ability to perform staggered workflow starts, among other things.

The disclosed implementations can process a number of samples using the system having a configuration of at least two assay bays. The disclosed implementations enable the common bay to be leveraged to quant, pool, denature, and/or dilute samples from multiple assay bays. In one example, the disclosed implementations enable different assays to be run in different assay bays in parallel.

### System

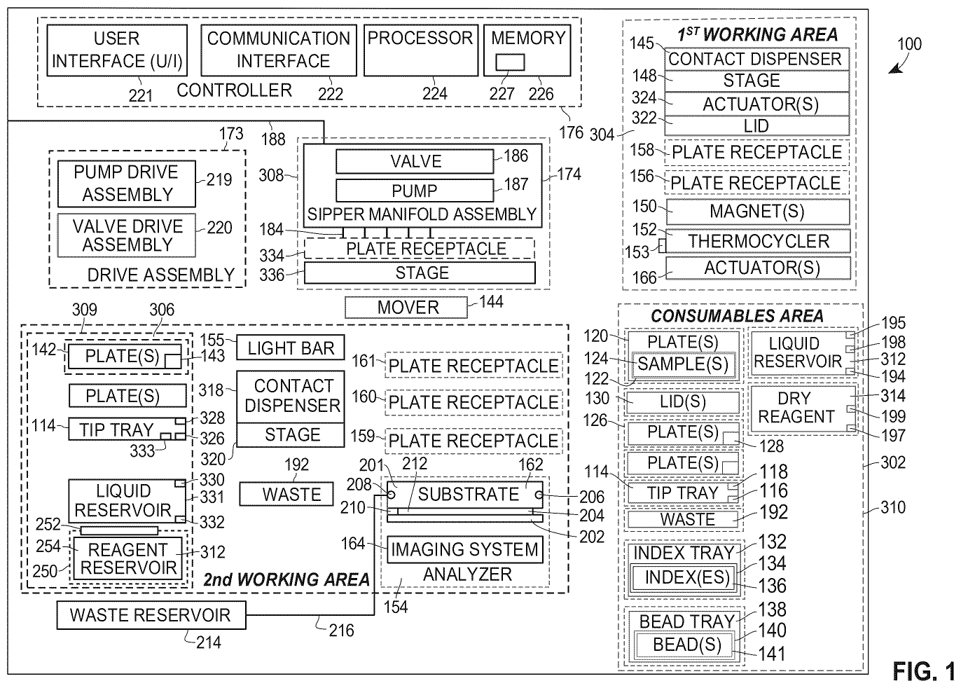

FIG. 1 illustrates a schematic diagram of an implementation of a system 100 for managing multiple parallel library preparation workflows with shared resources, in accordance with the teachings of this disclosure. The system 100 may be used to automatically, easily, and efficiently prepare DNA libraries for sequencing applications, for example. The system 100 may prepare the libraries when performing workflows such as whole genome sequencing workflows, DNA & RNA enrichment workflows, methylation workflows, split-pool amplicon workflows, amplicon workflows. The workflows may include one or more steps, such as amplification processes, cleanup processes, quantification processes, library normalization processes, pooling processes, denaturing processes, and/or diluting processes in some implementations.

The system may include a controller 176. The controller may be electrically and/or communicatively coupled to one or more components of the system 100 to perform various functions as described herein. The controller 176 may include a user interface 221, a communication interface 222, one or more processors 224, and a memory 226 storing instructions executable by the one or more processors 224 to perform the various functionalities described herein.

The user interface 221 may receive input from a user, and provide information to the user associated with the operation of the system 100 (e.g., information about the workflows being scheduled and/or taking place). The user interface 221 may include a touch screen, a display, a key board, a speaker(s), a mouse, a track ball, and/or a voice recognition system. The touch screen and/or the display may display a graphical user interface (GUI).

The system 100 may include the communication interface 222 to enable communication between the system 100 and one or more components, such as a remote system(s) (e.g., computers) using one or more network(s). The network(s) may include an intranet, a local-area network (LAN), a wide-area network (WAN), the intranet, etc. Some of the communications may be associated with workflows of the system 100, such as scheduling one or more workflows.

The system 100 may include one or more processors 224 and/or processor-based system(s) or a microprocessor-based system(s). In some implementations, the one or more processors 224 and/or the system 100 includes a reduced-instruction set computer(s) (RISC), an application specific integrated circuit(s) (ASICs), a field programmable gate array(s) (FPGAs), a field programmable logic device(s) (FPLD(s)), a logic circuit(s), and/or another logic-based device executing various functions including the ones described herein. The user interface 221, the communication interface 222, and the memory 226 may be electrically and/or communicatively coupled to the one or more processors 224.

The system 100 may include the memory 226. The memory 226 may include one or more of a hard disk drive, a flash memory, a read-only memory (ROM), erasable programmable read-only memory (EPROM), electrically erasable programmable read-only memory (EEPROM), a random-access memory (RAM), non-volatile RAM (NVRAM) memory, a compact disk (CD), a digital versatile disk (DVD), a cache, and/or any other storage device or storage disk in which information is stored for any duration (e.g., permanently, temporarily, for extended periods of time, for buffering, for caching, etc.).

The memory 226 may include a scheduler 227. The scheduler 227 may be stored as instructions on the memory 226 and executable by the processor 224. The scheduler 227 may include a common resource schedule which is used to schedule and/or execute (e.g., by the controller 176) one or more workflows. The scheduler 227 may receive a workflow request associated with one or more working areas 304, 306 (e.g., assay bay, common resource, etc.), and schedule the workflow to a common resource schedule. The common resource schedule may be used schedule and/or execute (e.g., via controller 176) one or more workflows which utilize the second working area 306 (e.g., common resource). The scheduler 227 or other suitable component (e.g., processor 224) may be configured to determine one or more conflicts in time and/or space with respect to the common resource/second working area 306 for at least two workflows associated with respective working areas 304 (e.g., a first and second assay bay), as further described herein. While the scheduler 227 is described as scheduling new workflows to a common resource schedule, in at least some aspects the scheduler 227 may also evaluate an existing workflow schedule and/or common resource schedule, to make optimizations and the like, as one having skill in the art will appreciate.

The system 100 may include one or more consumables areas 302, 309, a first working area 304, a second working area 306, and a loading area 308. In at least some embodiments, the first working area 304 may be referred to as an assay bay (e.g., first and second assay bays) and the second working area 306 may be referred to as a common resource.

The consumable areas 302 may be used to load and store reagents and consumables needed for a library preparation process, including, disposable tips, wet or dry assay specific reagents, wet or dry bulk reagents, and reaction plates and wells. The consumables area 302 may include a consumables receptacle 310 which may include a tip tray 114 having a first tip 116 and a second tip 118, a first plate 120 having a well 122 containing a sample 124, and a second plate 126 having a well 128. In at least some embodiments, one or more of the working areas 304, 306 may include a consumables area. For example, the second working area 306 includes consumables area 309, which may include a tip tray 114 and a third plate 142 having a well 143.

The consumables receptacle 310 may be a drawer that can be pulled out from the system 100 and loaded with the consumables 114, 116, 118, 120, 126. The consumables receptacle 310 may also include a lid 130, an index tray 132 having a well 134 containing indexes 136, a bead tray 138 having a well 140 containing beads 141, a liquid reservoir 312, and a dry reagent reservoir 314. One or more of these reagents 136 and/or 141 may be lyophilized and included with the dry reagent reservoir 314. In at least some aspects, one or more of the plates (e.g., plates 120, 126), the index tray 132, and/or the bead tray 138 may be stacked. The tip tray 114 may have a plurality of the first tips 116, a plurality of the second tips 118, and/or any suitable number of tips 116, 118. The tips 116, 118 may be different sizes, may be reusable for at least multiple portions of a workflow, as will be discussed in greater detail below.

The system 100 may include the first working area 304, which may include a contact dispenser 145, a stage 148, a magnet 150, and a thermocycler 152. The system 100 may include the second working area 306 having an analyzer area 154 (e.g., to analyze samples), a contact dispenser 318 and a stage 320, one or more of which can operate in and/or in conjunction with the first working area 304 and the second working area 306.

The contact dispenser 145 may be movable to aspirate/dispense liquid to the consumables area 302 and/or to the first working area 304. The contact dispenser 318 may be movable to aspirate/dispense liquid to the consumables area 302, to the first working area 304, and/or to the second working area 306. The contact dispenser 318 may carry one or more tips, e.g., to hold a volume of fluid.

The system 100 may include a mover 144, which in some aspects may be considered as part of the second working area 306. The mover 144 may include a robotic arm and/or include grippers. The stage 320 may carry the mover 144 and the contact dispenser 318 in some implementations. The mover 144 may include a gantry having grippers that can pick-and-place objects such as the plates 120, 126 and/or the trays 132, 138 between different areas 102, 304, 306, 308 of the system 100, e.g., the mover 144 may move the first plate 126 from the consumables area 302 to the first plate receptacle 156. The mover 144 may be implemented in different ways, however.

The stage 148 may be an x-z stage, such that the stage 148 is movable in the x and z directions (but not in the y direction). The stage 148 and the contact dispenser 148 may be movable to aspirate and/or dispense fluid between and above the consumables area 302 and the first working area 304 as a result. The contact dispenser 148 may, for example, move linearly in the x direction, which thereby reduces the risk of cross-contamination (between different samples) and allows some or all of the tips employed in the system 300 to be reusable for at least part of the processes performed by the system 300. The stage 148 may be implemented differently, however.

The stage 148 may align the contact dispenser 145 with the tip tray 114 and the contact dispenser 145 couples with the first tip 116 from the tip tray 114. While the contact dispenser 145 is mentioned coupling with one first tip 116, the contact dispenser 145 may couple with a number of the first tips 116 that corresponds to the number of the wells 122 in the first plate 120 and/or the number of the wells 122 in the first plate 120 containing the sample 124. The wells 122 may contain different samples 124, such as a biological sample derived from a human, animal, plant, bacteria, virus, or fungi. The first tip 116 may be a smaller pipette tip that is used to move smaller fluid volumes and the second tip 118 may be a larger pipette tip that is used to move larger fluid volumes, for example. Each of the first tip 116 and/or the second tip 118 may be exposed to a single sample during a workflow reducing the likelihood of cross-contamination and reducing the need to obtain a new tip after each operation. In some library preparation workflows, for example, each of the first tip 116 and/or the second tip 118 may be used through an entire workflow. The contact dispenser 145 may couple with and/or use different ones of the tips 116, 118 depending on the workflow and/or the processes within a workflow that the system 100 is implementing.

The stage 320 may be an x-y-z stage, such that the mover 144 is movable in the x, y, and z directions. The stage 320 and the contact dispenser 318 may be movable to aspirate and/or dispense fluid between and above the consumables area 302, the first working area 304, and/or the second working area 306 as a result. The contact dispenser 148 may, for example, move linearly in the x direction, which thereby reduces the risk of cross-contamination (between different samples) and allows some or all of the tips employed in the system 100 to be reusable for at least part of the processes performed by the system 100. The stage 148 may be implemented differently, however.

The second working area 306 may include a light bar 155 that may be used to degrade oligonucleotides, such as a high power ultraviolet light (UV) light bar that is regularly used throughout a workflow to repeatedly degrade oligonucleotides to deter cross contamination in some implementations.

The first working area 304 may include a first plate receptacle 156 and a second plate receptacle 158, the second working area 306 may include a third plate receptacle 159, a fourth plate receptacle 160, and a fifth plate receptacle 161, and the analyzer area 154 may include a substrate 162 and an imaging system 164, in at least some implementations. For example, the analyzer area 154 may use the substrate 162 that is implemented by a well plate in which a portion of the sample and a dye are dispensed, and the imaging system 164 may image the portion of the sample in the well plate to determine a concentration of the sample.

The second working area 306 may include a reagent receptacle 250 having an access opening 252. A reagent reservoir 254 is shown received within the reagent reservoir 306. The first working area 304 may additionally or alternatively include a reagent receptacle 250 having an access opening 252. The reagent receptacle 250 may be refrigerated and may be a drawer that can be pulled out from the system 100 and loaded with the reagent reservoir 254. The reagent reservoir 254 may be accessed through the access opening 252 by the contact dispenser 318 to aspirate reagent from the reagent reservoir 254, for example.

The loading area 308 may be associated with loading and/or transferring a prepared sample to a system such as a sequencing system. For example, the first working area 304 may be associated with amplification processes and cleanup processes and the second working area 306 may be associated with quantification processes, library normalization processes, pooling processes, denaturing processes, and/or diluting processes. The loading area 308 may include a sipper manifold assembly 174 in the implementation shown. The sipper manifold assembly 174 may include sippers 184 to couple the sipper manifold assembly to a corresponding number of the flow cells. The sipper manifold assembly 174 may include a valve 186 to control the flow of fluid through a fluidic line 188. The sipper manifold assembly 174 may include a pump 187 to selectively flow the prepared sample from a well 128, 143 through the sipper 184, through the fluidic line 188, and out of the system 100, e.g., to another system used to perform an analysis on one or more samples of interest.

The valve 186 may be implemented by a rotary valve, a pinch valve, a flat valve, a solenoid valve, a check valve, a piezo valve, etc. Other fluid control devices may prove suitable. The pump 187 may be implemented by a syringe pump, a peristaltic pump, and/or a diaphragm pump. Other types of fluid transfer devices may be used, however. The controller 176 is electrically and/or communicatively coupled to components of the system 100 to perform various functions as disclosed herein. The sipper manifold assembly 174 may alternatively be omitted.

The actuator 166 can move the magnet 150 between an upward position where the magnet 150 affects any plate positioned on the first plate receptacle 156 and a downward position where the magnet 150 does not affect any plate positioned on the first plate receptacle 156. The magnet 150 being moved relative to the first plate receptacle 156 and any plate 120, 126, 142 positioned on the first plate receptacle 156 allows less area on the first working area 304 to be consumed. The magnet 150 can moreover be moved with relatively higher confidence as compared to an alternative approach to moving one of the plates 120, 126, 142 filled with samples to a separate magnet station. The magnet 150 may be implemented by a halbach array configuration to strengthen and focus the corresponding magnetic fields.

### Amplification Processes

In one example workflow, the stage 148 aligns the contact dispenser 145 with the index tray 132 and the contact dispenser 145 aspirates the indexes 136 from the index tray 132 using the first tip 116. The stage 148 can then align the contact dispenser 145 with the first plate 120 and the contact dispenser 145 dispenses the indexes 13 into the well 122 of the first plate 120. The mover 144 moves the lid 130 from the consumables area 302 and places the lid 130 on the first plate 120 to cover the well 122 of the first plate 120 with the lid 130. The first working area 304 also includes a lid 322 and an actuator 324. The lid 322 may be referred to as a cover and/or a door. The actuator 324 may move the lid 322 in operation relative to the plate receptacle 156 to cover the plate receptacle 156 during amplification processes, for example. The lid 322 may thus be positioned to enclose the first plate 120 during the amplification processes. The lid 130 and/or the lid 322 may enclose the first plate 120 during the amplification processes.

The thermocycler 152 is aligned with the first plate receptacle 156 and the thermocycler 152 amplifies the sample 124 within the well 122 of the first plate 120. The thermocycler 152 and/or the magnet 150 can act on a plate 120, 126 received at the first plate receptacle 156 in the implementation shown. The thermocycler 152 may alternatively be spaced from the magnet 150.

The actuator 324 can move the lid 322 off of the first plate 120 and/or the mover 144 can move the lid 130 from the first plate 120 to the consumables area 302 after the amplification processes are complete. The consumables area 302 includes a waste 192 that can receive used consumables such as, for example, the lid 130. The lid 130 may alternatively be reused, however. The waste 192 may be a waste tray having an absorbent material to absorb liquid waste.

### Cleanup Processes

The system 100 may perform cleanup processes, according to one or more workflows, after the amplification processes are performed. The stage 148 aligns the contact dispenser 145 with the bead tray 138 and the contact dispenser 145 aspirates the beads 141 from the bead tray 138. The contact dispenser 145 may aspirate the beads 141 using the same first tip 116 used to aspirate the indexes 136. The contact dispenser 145 may alternatively use another one of the first tips 116 or one of the second tips 118 to aspirate the beads 141.

The stage 148 aligns the contact dispenser 145 with the first plate 120 and the contact dispenser 145 dispenses the beads 141 into the well 140 of the first plate 120 as part of the cleanup process. The stage 148 aligns the contact dispenser 145 with the liquid reservoir 312 and the contact dispenser 145 aspirates first reagent 194 from the liquid reservoir 312. The stage 148 then aligns the contact dispenser 145 with the first plate 120 and the contact dispenser 145 dispenses the first reagent 194 into the well 122 of the first plate 120. The contact dispenser 145 may alternatively aspirate hydrating liquid 195 from the liquid reservoir 312 and then dispense the hydrating liquid 195 into dried reagent 197 contained within the dry reagent reservoir 314 to rehydrate the dried reagent 197 and form the first reagent 194. The contact dispenser 145 may pipette mix the dried reagent 197 and the hydrating liquid 195. The first reagent 194 may be a bead buffer and the sample 124 may bind to the beads 141 in the presence of the bead buffer. The contact dispenser 145 may be able to jet dispense with adequate liquid velocity to enable jet mixing in some implementations. The system 100 may also include a shaker to enable mixing.

The stage 148 aligns the contact dispenser 145 with the tip tray 114 and the contact dispenser 145 places the first tip 116 in the tip tray 116 and the contact dispenser 145 then couples with the second tip 118 from the tip tray 114. While the contact dispenser 145 is mentioned coupling with one second tip 118, the contact dispenser 145 may couple with a number of the second tips 118 that corresponds to the number of the wells 122 in the first plate 120 and/or the number of the wells 122 in the first plate 120 containing the sample 124.

The actuator 166 moves the magnet 150 toward the plate receptacle 156 and the magnet 150 draws the beads 141 toward the magnet 150. The beads 141 and the sample 124 bound to the beads 141 may be positioned toward the bottom of the well 122 of the first plate 120 or on a side(s) of the well 122. The tips 116 and/or 118 may easily access the well 122 if the beads 141 are on the side of the well 122. The magnet 150 may cause the beads 141 to be in any position within the well 122, however.

The stage 148 aligns the contact dispenser 145 with the first plate 120 and the contact dispenser 145 aspirates the first reagent 194 from the well 122 of the first plate 120. The contact dispenser 145 may dispense the first reagent 194 aspirated from the well 122 of the first plate 120 into the waste 192.

The stage 148 aligns the contact dispenser 145 with the liquid reservoir 312 and the contact dispenser 145 aspirates second reagent 198 from the liquid reservoir 312 and the stage 148 then aligns the contact dispenser 145 with the first plate 120 and the contact dispenser 145 dispenses the second reagent 198 into the well 122 of the first plate 120. The contact dispenser 145 may alternatively aspirate hydrating liquid 195 from the liquid reservoir 312 and then dispense the hydrating liquid 195 into a dried reagent 199 contained within the dry reagent reservoir 314 to rehydrate the dried reagent 199 and form the second reagent 198. The second reagent 198 may be an elution buffer that releases the sample 124 from being bound to the beads 141 and, specifically, releases DNA associated with the sample 124 from being bound to the beads 141.

The mover 144 moves the second plate 126 from the consumables area 302 to the second plate receptacle 158. The system 100 can use the second plate 126 for a transfer operation. The second plate 126 may alternatively remain in the consumables area 302 during the transfer operation. The actuator 166 moves the magnet 150 toward the second plate receptacle 158 to draw the beads 141 toward the magnet 150 and, thus, provide a substantially bead-free eluate solution comprising the second reagent 198 and the sample 124 within the well 122.

The stage 148 aligns the contact dispenser 145 with the first plate 120 and the contact dispenser 145 aspirates the second reagent 198 and the sample 124 from the well 122 of the first plate 120 using the second tip 118, for example. The stage 148 aligns the contact dispenser 145 with the second plate 126 and the contact dispenser 145 dispenses the second reagent 198 and the sample 124 into the well 128 of the second plate 126. The second plate 126 may alternatively be positioned in the consumables area 302 when the contact dispenser 145 dispenses the second reagent 198 and the sample 124 into the well 128 of the second plate 126. The second plate receptacle 158 may be omitted from the second working area 306 in such implementations.

### Quantification Processes

The system 100 may perform the quantification processes, according to one or more workflows, after the cleanup processes are performed. The mover 144 moves the second plate 126 from the first working area 304 to the plate receptacle 159 of the second working area 306 to initiate the quantification processes in some implementations. The mover 144 may alternatively move the second plate 126 from the consumables area 302 to the plate receptacle 159 of the second working area 306 to initiate the quantification processes in implementations when the second plate 126 remains in the consumables area 302 during the transfer operations. The stage 320 aligns the contact dispenser 318 with the tip tray 114 of the second working area 306 in some implementations and the contact dispenser 145 couples with a tip 326 from the tip tray 114.

The substrate 148 may be a plate having a well. The substrate may be a consumable that is disposed of after use. The imaging system 164 may be spaced from the substrate 148 and coupled to a portion of the system 100 such as a frame of the system 100. The imaging system 164 may alternatively be carried by a stage.

The stage 320 aligns the contact dispenser 318 with the second plate 126 to perform the quantification processes and the contact dispenser 318 aspirates a portion of the second reagent 198 and the sample 124 from the well 128 of the second plate 126. The portion of the second reagent 198 and the sample 124 may be about 2 μL.

The stage 320 aligns the contact dispenser 318 with the substrate 162 and the contact dispenser 318 dispenses the portion of the second reagent 198 and the sample 124 into a well of the substrate 162 as an example. A dye may also be dispensed into the well of the substrate 162 by the contact dispenser 318. The imaging system 164 obtains image data of the portion of the second reagent 198 and the sample 124 within the well of the substrate 162. The imaging system 164 and/or the system 100 uses the image data to determine a concentration of the sample 124. The mover 144 may move the substrate 162 to the waste 192 of the consumables area 309 of the second working area 306.

The substrate 162 may alternatively be implemented by a pair of plates 201, 202 between which a gap 204 is defined. The substrate 162 in such an implementation includes an inlet 206 and an outlet 208 in fluid communication with the gap 204 and a seal 210 positioned between the pair of plates 201, 202. The plates 201, 202 and the seal 210 define a channel 212 between the inlet 206 and the outlet 208. A waste reservoir 214 may be fluidly coupled to the outlet 208 of the substrate 162 by a fluidic line 216.

In the alternative implementation of the substrate 162, the stage 320 aligns the contact dispenser 318 with the inlet 206 of the substrate 162 and the contact dispenser 318 dispenses the portion of the second reagent 198 and the sample 124 into the inlet 206 of the substrate 162. The portion of the second reagent 198 and the sample 124 may flow and/or be positioned between the inlet 206 and the outlet 208 in this implementation and the imaging system 164 obtains image data of the portion of the second reagent 198 and the sample 124. The imaging system 164 and/or the system 100 uses the image data to determine a concentration of the sample 124. Negative pressure, oil, and/or another substance may be used to urge the portion of the second reagent 198 and the sample 124 between the inlet 206 and the outlet 208. The first plate 201 may alternatively be hingably coupled or removably coupled to the second plate 202 to allow the contact dispenser 318 to dispense the portion of the second reagent 198 and the sample 124 onto the second plate 202 prior to the first plate 201 being positioned overtop of the second plate 202. The system 100 may perform quantification processes in different ways, however.

### Normalization Processes

The system 100 may perform the normalization processes, according to one or more workflows, after the quantification processes are performed. The stage 320 aligns the contact dispenser 318 to initiate the normalization processes in some implementations. The contact dispenser 318 aspirates a diluent 330 from a liquid reservoir 331 of the second working area 306. The stage 320 then aligns the contact dispenser 318 with the second plate 126 and the contact dispenser 318 dispenses the diluent 330 into the well 128 of the second plate 126 to dilute the sample 124 based on the concentration of the sample determined. The sample 124 within the well 128 of the second plate 126 will have a concentration within a threshold value after the diluent 330 is added to the well 128 as a result. The diluent 330 may be a buffer.

### Pooling Processes

The system 100 may perform the pooling processes, according to one or more workflows, after the quantification processes are performed. The stage 320 aligns the contact dispenser 318 with the tip tray 114 of the second working area 306 and the contact dispenser 318 places the tip 326 in the tip tray 114 and the contact dispenser 318 then couples with another tip 328 from the tip tray 114 to initiate the pooling processes in some implementations. The mover 144 moves a plate 142 from the second working area 306 to the plate receptacle 160 of the second working area 306. The stage 320 aligns the contact dispenser 318 with the second plate 126 and the contact dispenser 318 aspirates the sample 124 from the well 128 of the second plate 126. The stage 320 then aligns the contact dispenser 318 with the third plate 142 and the contact dispenser 318 dispenses the sample 124 into the well 143 of the third plate 142. Additional samples from other wells of the second plate 126 may be deposited into the well 143 of the third plate 142 in a similar manner to combine a plurality of normalized samples together. A single tip can be used for the pooling processes. The contact dispenser 318 may pipette from final archive library well directly to pool and, thus, unique tips per sample may be used.

### Denaturing Processes

The system 100 may perform the denaturing processes, according to one or more workflows, after the pooling processes are performed (though in some implementations, denaturing need not be performed). The stage 320 aligns the contact dispenser 318 with the tip tray 114 of the second working area 306 and the contact dispenser 318 places the tip 328 in the tip tray 114 and the contact dispenser 318 then couples with another tip 333 from the tip tray 114 to initiate the denaturing processes in some implementations. The contact dispenser 318 may use the same tip 328 used during the pooling processes in some implementations, however. The stage 320 aligns the contact dispenser 318 with the liquid reservoir 331 and the contact dispenser 318 aspirates reagent 332 from the liquid reservoir 331. The stage 320 then aligns the contact dispenser 318 with the third plate 142 and the contact dispenser 318 dispenses the reagent 332 into the well 143 of the third plate 142 to denature the pooled and normalized samples. The reagent 332 may be Sodium hydroxide (NaOH). Other denaturing processes may prove suitable. For example, Formamide or an equivalent may be used during the denaturing processes.

### Diluting Processes

The system 100 may dilute the pooled and denatured samples, according to one or more workflows, after the pooling and the denaturing processes are performed. The contact dispenser 318 aspirates a diluent 330 from a liquid reservoir 331 of the second working area 306. The stage 320 then aligns the contact dispenser 318 with the third plate 142 and the contact dispenser 318 dispenses the diluent 330 into the well 128 of the third plate 142 to dilute the sample 124 based on the concentration of the sample based on specifications of the sequencing system (e.g., the system 900) into which the sample is to be loaded. The pooled samples 124 within the well 143 of the third plate 142 will have a concentration within a threshold value after the diluent 330 is added to the well 143 as a result. The diluent 330 may be a buffer.

### Loading Processes

The system 100 may perform the loading processes, according to one or more workflows, after the denaturing processes and/or after the diluting processes are performed. The mover 144 moves the third plate 142 from the second working area 306 to a plate receptacle 334 of the loading area 308. The loading area 308 is shown including a stage 336 that can be used to move the plate receptacle 334 relative to the sipper manifold assembly 174. The sipper manifold assembly 174 flows the denatured samples from the well 128, 143 through the corresponding sipper(s) 184, through the fluidic line(s) 188, and out of the system 100 to another system for sequencing.

The system 100 also includes a drive assembly 173. The drive assembly 173 includes a pump drive assembly 219 and a valve drive assembly 220. The pump drive assembly 219 may be adapted to interface with the pump 187 to pump fluid from the reagent reservoir 110 to the non-contact dispenser 146. The valve drive assembly 220 may be adapted to interface with the valve 186 to control the position of the valve 186.

In some aspects, more or fewer instances of the various components of the system 100 shown in FIG. 1 may be included in the system 100 (e.g., four working areas 304, three consumable areas 310, etc.).

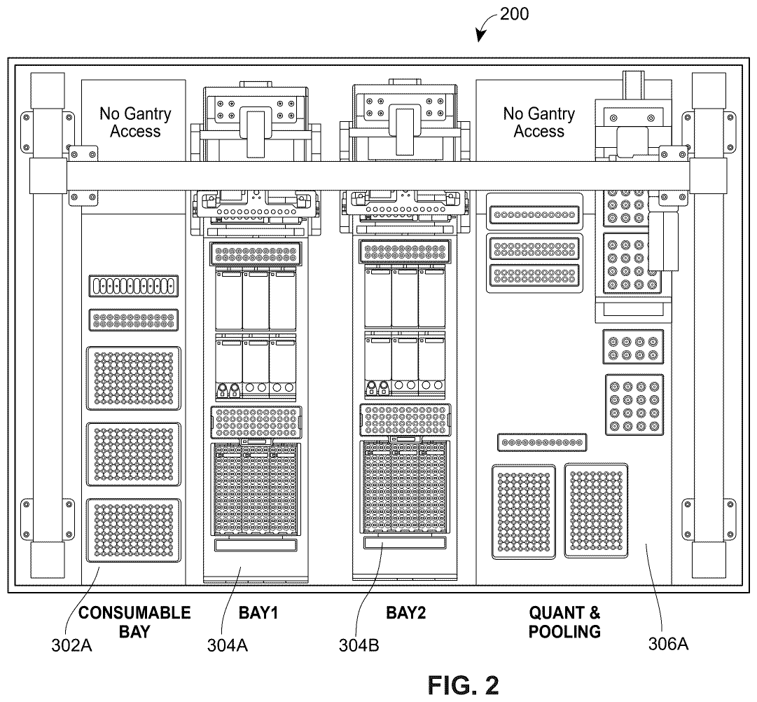

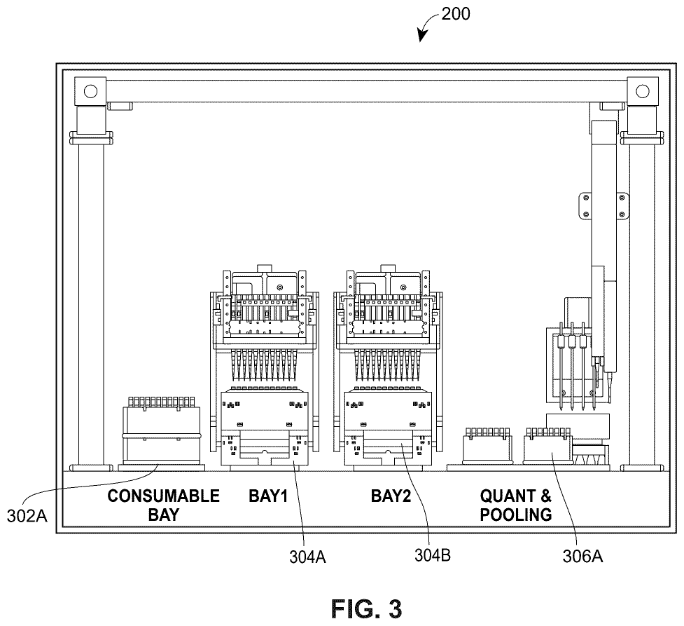

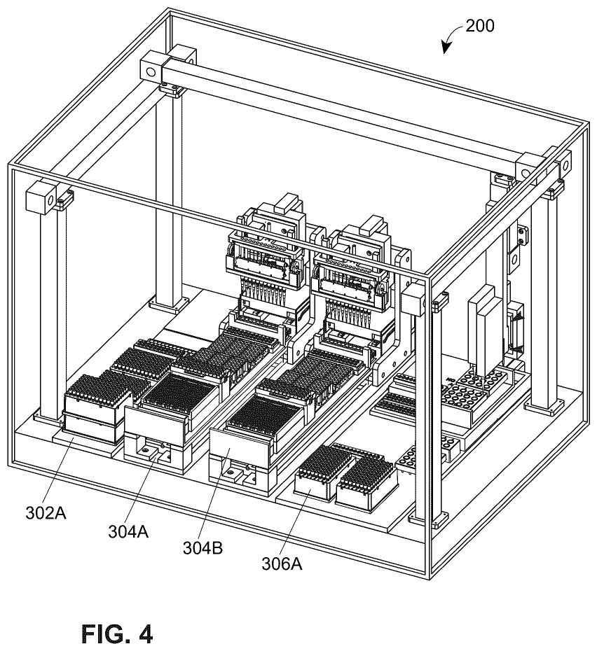

FIG. 2 is a top view of a system 200 that can be used to implement the system 100 of FIG. 1. The system 200 of FIG. 2 includes a consumables areas 302A such as consumable area 302, a first assay bay 304A such as working area 304, a second assay bay 304B such as second working area 304, and a common resource 306A such as common resource 306. FIG. 3 is a front view of the system 200, and FIG. 4 is an isometric view of the system 200.

### Scheduling

As should be understood by now, one or more of the systems 100, 200, may carry out workflows to prepare a library of samples. The workflows may use and/or include at least two assay bays such as the assay bays 304A, 304B, a common resource such as the common resource 306A, and/or a scheduler such as the scheduler 227. At one or more times, the scheduler may schedule workflows to the common resource schedule using one or more rules and/or workflow characteristics to resolve conflicts over the common resource, which may further optimize operation of the system 100, 200, improve the overall time to complete one or more workflows, etc. The workflow characteristics may be indicated by the workflow (e.g., as metadata of the workflow request) or manually provided by a user of the system 100 (e.g., via the user interface 221). Additionally or alternatively, the scheduler 227 or any other suitable component of system 100 (e.g., processor 224) may determine the workflow characteristics, and/or the workflow characteristics may obtained in any other suitable manner.

The scheduler 227 may schedule the workflows according to one or more of the following methods: 1) First come, first serve/wait until available, 2) Prioritization, 3) Resource stealing, 4) Resource Future Reservation, 5) Spatial Resource Discretization, 6) Spatial Resource Collision Detection, 7) Pre-allocated Resource Time, and/or 8) Parallel Operation Start/End Flexibility. These methods are described in more detail below.

In at least some aspects, the workflow characteristics may include temporal characteristics such as a minimum and/or maximum wait time which may be added to at least a portion of a workflow to delay execution of the workflow. For example, if a workflow is scheduled (or request to be scheduled) to the common resource schedule to begin execution at 12:00 pm and its workflow characteristics indicate it has a maximum wait time of 20 minutes, the scheduler (e.g., scheduler 227) may schedule the workflow on the common resource schedule by up to 20 minutes after its proposed start time, i.e., 12:20 pm, to resolve a conflict. In at least some aspects, the workflow characteristics may indicate a minimum and/or maximum run time for at least a portion of the workflow, a proposed scheduled time for at least a portion of the workflow, and/or a priority level of the workflow.

For example, if two workflows create a conflict if scheduled to the common resource schedule at the proposed scheduled times indicated in each workflow's characteristics (e.g., via the workflow request), the scheduler 227 may schedule a higher priority workflow to the common resource schedule before the lower priority workflow to resolve the conflict.

In at least some aspects, the workflow characteristics may include spatial characteristics such as the location of a target object (e.g., a consumable), the location of a movable assay bay component (e.g., contact dispenser 145), and/or the location of a movable common resource component (e.g., the mover 144). For example, a spatial conflict may exist between two workflows due to a contact dispenser and mover of each workflow utilizing the same space or zone of the system 100 200 at the same time. The scheduler may determine that, based upon the location of the conflicting moveable components, adding a wait time to the workflow of the contact dispenser will resolve the spatial conflict by delaying when the contact dispenser enters the zone where the conflict occurs, and schedule the contact dispenser workflow to the common resource schedule with a delay to resolve the conflict.

In one embodiment, the scheduler such as scheduler 227 receives a first workflow scheduling request for a first workflow associated with the first assay bay (e.g., first working area 304) which utilizes the common resource (e.g., second working area 306). For example, a user desiring to carry out a workflow may submit the workflow request via the user interface 221 of the system 100. The workflow may be and/or include one or more processes such as an amplification processes, cleanup processes, quantification processes, library normalization processes, pooling processes, denaturing processes, diluting processes, and/or any other suitable process(es). The scheduler may also receive a second workflow request for a second workflow associated with the second assay bay (e.g., first working area 304) which utilizes the common resource. The scheduler may determine that the first and second workflows conflict in time and/or space with respect to the common resource. For example, both workflows may conflict when needing the mover of the common resource at the same time, when needing the common resource to conduct processes at the same time, when the mover and contact dispenser need to occupy the same space at the same time, or any other suitable conflict between workflows. The scheduler may obtain one or more workflow characteristics of the first and second workflows, e.g., by analyzing metadata of the workflow request, analyzing the steps of the workflow, from historical information associated with the same/similar workflow which may be stored in memory such as memory 226, or any other suitable manner of obtaining workflow characteristics. The scheduler may assign the first and second workflows to the common resource schedule based on the workflow characteristics of the first and second workflows, and use one or more rules which resolve the conflict between the first and second workflows.

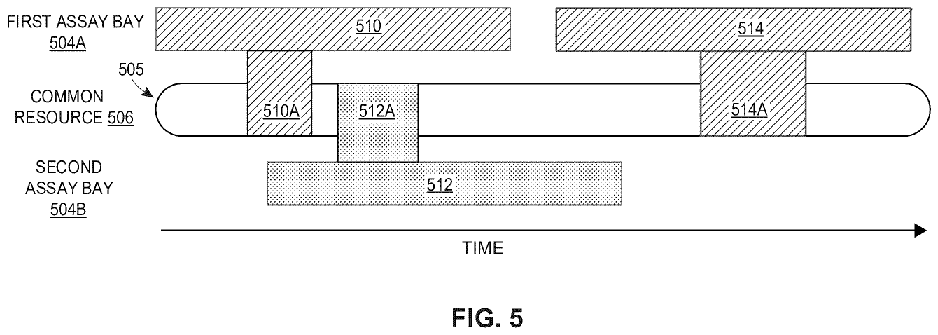

FIG. 5 is an example block diagram illustrating scheduled workflows for a first assay bay 504A and a second assay bay 504B each utilizing the common resource 506. The scheduler may receive a first workflow request for a first workflow 510 associated with the first assay bay 504A (e.g., a workflow for an amplification process), receive a second workflow request for a second workflow 512 associated with the second assay bay 504B (e.g., a workflow for a cleanup process), and a third workflow request for a third workflow 514 associated with the first assay bay 504A (e.g., a workflow for a quantification process). The system 100, 200 may receive the workflow requests, which may be electronic requests including data indicating a “recipe” or steps of the workflow, from another device (e.g., computer, web interface, etc.) via communication interface 222, may receive the workflow requests from a user of the system 100, 200 via the user interface 221, or in any other suitable manner. Portions (e.g., one or more steps) 510A, 512A, 514A of the first workflow 510, second workflow 512, and third workflow 514 respectively may request and/or utilize the common resource 506, as indicated in FIG. 5 via portions 510A, 512A, 514A overlapping the horizonal bar 505 associated with the schedule and/or utilization of the common resource 506. As none of the portions 510A, 512A, 514A of the workflows 510, 512, 514 request to utilize the common resource at the same time, no conflicts arise over the common resource 506. Thus, the scheduler 227 may assign the workflows 510, 512, 514 to the common resource schedule in the order in which the workflows 510, 512, 514 are received according to first come, first served rule. This may include scheduling the first workflow 510 to the common resource schedule first, the second workflow 512 to the common resource schedule next, and the third workflow 514 to the common resource schedule after that. The controller 176 may then execute the workflows according to the common resource schedule.

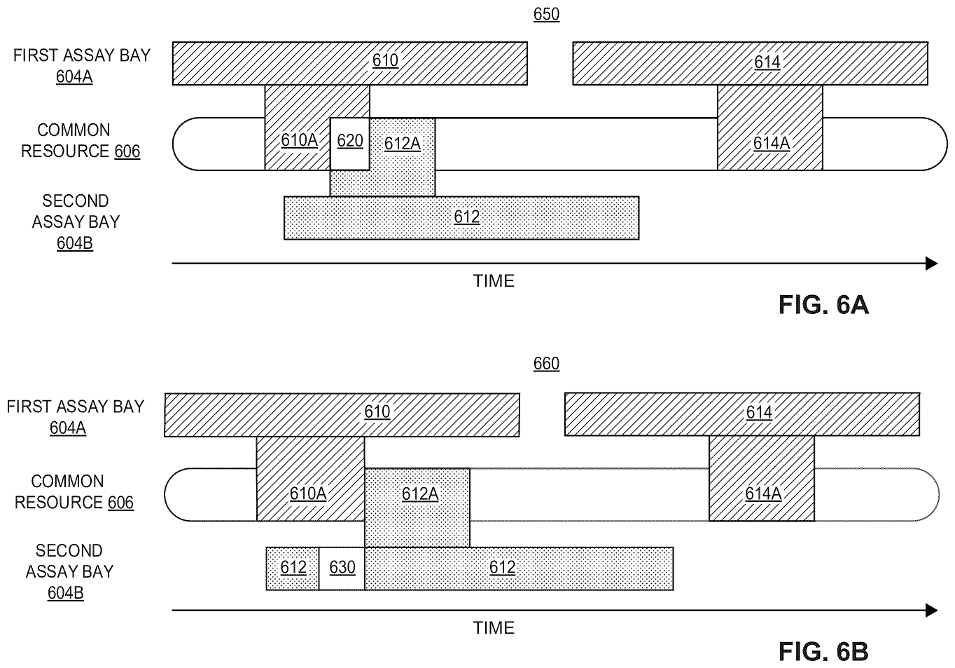

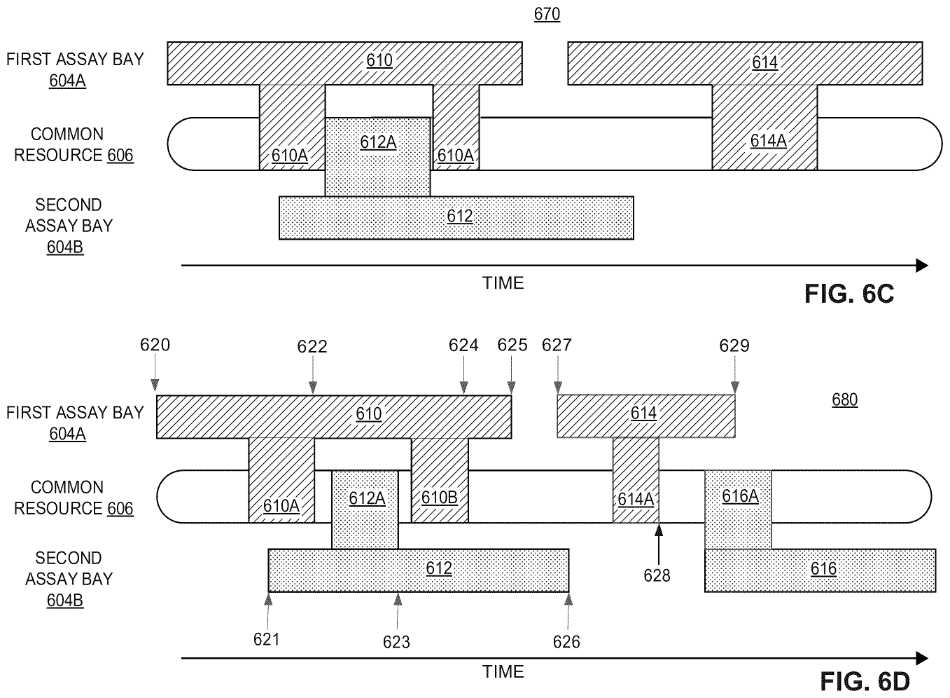

FIG. 6A is an example block diagram illustrating scheduled workflows which include a conflict for a first assay bay 604A and a second assay bay 604B each utilizing the common resource 606, according to the scenario 650. The first workflow 610 is associated with the first assay bay 604A, the second workflow 612 is associated with the second assay bay 604B, and the third workflow 614 is also associated with the first assay bay 604A. Portions (e.g., one or more steps) 610A, 612A, 614A of the first workflow 610, second workflow 612 and third workflow 614 respectively, request the common resource 606 such that a conflict 620 arises over the common resource 606 for portion 610A of the first workflow 610 and portion 612A of the second workflow 612. The conflict 620 is due to both the first assay bay 604A and second assay bay 604B requesting the common resource 606 during the same time period for their respective portions 610A, 612A. For example, the conflict 620 may be due to the workflow 610 and workflow 612 both requesting the mover (e.g., mover 144) of the common resource 606 to pick-and-place a plate during the time associated with conflict 620.

### First Come First Served/Wait Time

In a first example, to resolve the conflict 620 of FIG. 6A the scheduler may add a wait time as illustrated by the example block diagram of FIG. 6B. The block diagram 660 of FIG. 6B illustrates scheduled workflows for a first assay bay 604A and a second assay bay 604B which resolve a conflict using a wait time rule, according to the scenario 660. To schedule the workflows 610, 612 in a manner which resolves and/or avoids the conflict 620, the scheduler may obtain workflow characteristics for one or more of the workflows requesting to be scheduled, such as the first and second workflows 610, 612.

### Prioritization

For example, the characteristics may indicate a priority, e.g., workflow characteristics of workflow 610 may indicate a higher priority which require it to take place before the second workflow 612 having workflow characteristics indicating a lower priority. The second workflow 612 may have additional workflow characteristics indicating it can tolerate adding a wait time to the workflow 612. To resolve the conflict 620 based upon the workflow characteristics, the scheduler may schedule the workflows to the common resource schedule by applying a rule which adds a wait time 630 between to the start of portion 612A requesting the common resource, alleviating the conflict 620. Adding the wait time 630 allows the portion 610A of the first workflow 610 to finish using the common resource before the portion 612A begins using the common resource, while honoring the higher priority workflow characteristics of workflow 610 with respect to the lower priority workflow characteristics of workflow 612.

Further, as the scheduler is scheduling the workflows 610, 612 to the common resource schedule in the order in which the requests are received, the scheduler may also apply the first come, first served rule. The controller 176 may then execute the workflows according to the common resource schedule.

### Resource Stealing

In a second example, to resolve the conflict 620 of FIG. 6A the scheduler may steal the common resource, as illustrated by the example block diagram of FIG. 6C. The block diagram 670 of FIG. 6C illustrates scheduled workflows for a first assay bay 604A and a second assay bay 604B which resolve a conflict using a resource stealing rule, according to the scenario 670. In this example, the workflow characteristics of the second workflow 612 may indicate a higher priority than the first workflow 610, such that that the portion 612A cannot tolerate a wait. For example, the mover of common resource 606 may be required to deliver a tray for a time sensitive process step of workflow portion 612A. The characteristics of the first workflow 610 may indicate that it can tolerate a wait of up to 20 seconds in between the start and stop of portion 610A. To resolve the conflict 620, the scheduler may assign the workflows to the common resource schedule by applying a rule allowing the higher priority second workflow 612 to steal the common resource 606 from the lower priority workflow 610 during the time requested by the portion 612A to access the common resource 606. This would allow workflow 612 to steal the common resource long enough to deliver the tray for workflow portion 612A, and still free up the common resource 606 thereafter to complete workflow portion 610A. The controller 176 may then execute the workflows according to the common resource schedule.

### Future Scheduling

In at least some embodiments, the scheduler may receive a workflow request which includes a proposed future time for the workflow utilizing the common resource. The scheduler may determine that the common resource is available at the proposed time and assign the future workflow to the common resource schedule at the proposed time. However, if there is a conflict at the proposed future time, the scheduler may apply one or more rules to resolve the conflict in light of the workflow characteristics.

In at least some aspects, the scheduler may receive a new workflow request requesting the common resource during a time in which existing workflows are already scheduled and/or in-progress. In such a scenario, the scheduler may determine an open time on the common resource schedule during which the new workflow associated with the new workflow request may be scheduled. The open time may be an available time due to adding a wait time to an existing workflow, stealing a resource from an existing workflow, scheduling a workflow at a time when no other workflows are scheduled, or any other suitable open time. The scheduler may determine the open time for the new workflow at one or more times during an existing and/or in-progress workflow. For example, an existing workflow may include multiple steps and the scheduler may determine an open time after each step is executed, after the completion of the entire workflow, or at any other suitable time.

Turning to the example block diagram of FIG. 6D, the block diagram illustrates scheduled workflows for a first assay bay 604A and a second assay bay 604B which resolve a conflict using a future scheduling open time rule. At times 620-629, the scheduler may determine whether an open time slot exists and/or may be created on the common resource schedule to schedule a new workflow, such as at the start of a workflow (e.g., times 620, 621, 627 associated with workflows 610, 612, 614 respectively), at the end of a workflow step (e.g., times 622, 623, 624, 628 associated with workflow portions 610A, 612A, 610B and 614B respectively), at the end of a workflow (e.g., times 625, 626, 629 associated with workflows 610, 612, 614 respectively), and/or any other suitable time. When an open time slot is available or created (e.g., by resource stealing, adding a wait time, etc.), the scheduler may schedule the new workflow of the new workflow request to the common resource schedule, such as scheduling workflow 616 to the common resource schedule. The controller 176 may then execute the workflows according to the common resource schedule.

### Pre-Allocation

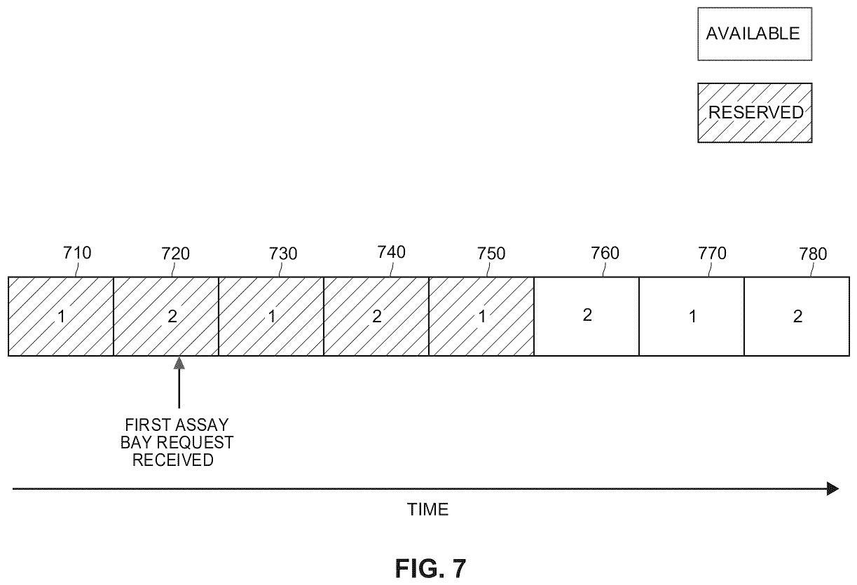

In at least some aspects, the scheduler may pre-allocate blocks of time on the common resource schedule for each assay bay to utilize the common resource. For example, a first assay bay such as assay bay 604A may be pre-allocated to the common resource schedule at one hour intervals for hours having an even number (e.g., 2:00; 4:00; 6:00, etc.) and a second assay bay such as assay bay 604B may be pre-allocated to the common resource schedule at one hour intervals for hours having an odd number (e.g., 1:00; 3:00; 5:00, etc.). In response to receiving a workflow request from an assay bay, the scheduler may assign at least a portion of the workflow of the request to the common resource schedule at a next-available pre-allocated block of time for that assay bay. FIG. 7 is an example block diagram illustrating a pre-allocated workflow schedule. The common resource schedule has blocks 710, 730, 750, 770 pre-allocated to a first assay bay and blocks 720, 740, 760, 780 pre-allocated to a second assay bay. The scheduler may receive a workflow request utilizing the common resource from the first assay bay during block 720. The next available pre-allocated block for the first assay bay which is not already reserved on the common resource schedule for another workflow is unshaded/available block 770. The shading indicates a pre-allocated block which is already reserved for an assay bay's workflow. Accordingly, the scheduler may schedule the first assay bay workflow request for the common resource to block 770 on the common resource schedule to avoid any conflicts. The controller 176 may then execute the workflows according to the common resource schedule.

### Spatial Conflicts

The systems 100, 200 may include one or moveable components, as previously described. For example, one or more assay bays may include a movable contact dispenser, such as contact dispenser 145, 318, which may be linearly movable in a first direction parallel to at least one of the assay bays. In another example, the common resource may include a mover, such as mover 144, which may be movable in at least the first direction parallel to the assay bays and at least a second direction perpendicular to the first direction. For example, the mover may be movable along a gantry system such as the gantry system described above and illustrated in FIGS. 2-4. In at least some aspects, the mover may also be movable in an upward and downward direction (e.g., along a z-axis).

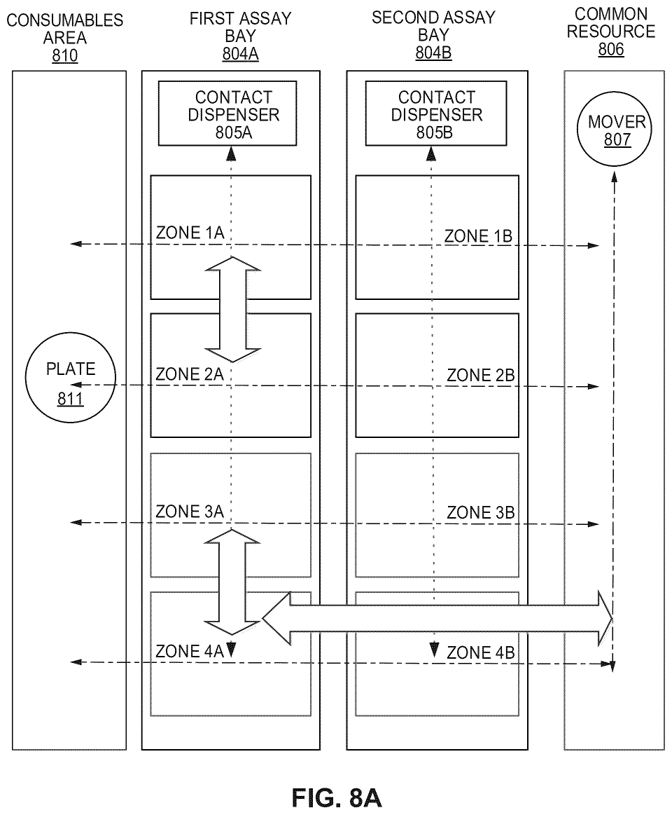

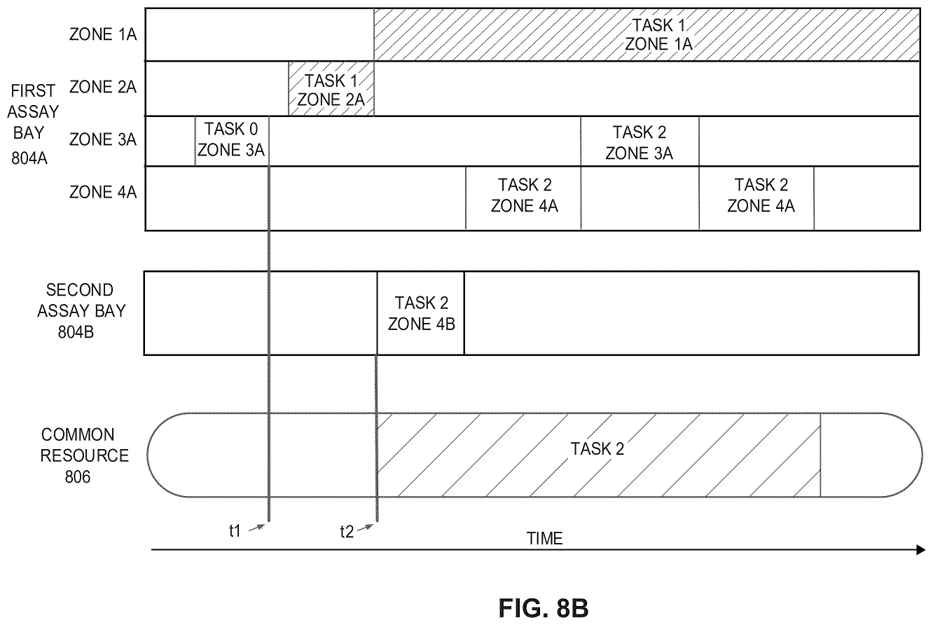

In at least some embodiments, the system 100, 200 may include one or more spatial zones representing physical spaces which one or more movable components may traverse or otherwise move into, out of, and/or through during a workflow. The example block diagram of FIG. 8A illustrates spatial zones of the system 100, 200. In this example, a first assay bay 804A such as assay bay 604A, and second assay bay 804B such as assay bay 604B each include spatial zones 1A-4A and 1B-4B respectively.

The contact dispenser 805A, 805B of the assay bays 804A, 804B may be controllable via a controller such as controller 176 and may be linearly movable in a first direction parallel to the assay bays 804A, 804B and across all four spatial zones of the respective assay bay. The common resource mover 807 may be controllable via the controller. The common resource mover 807 may also be operatively coupled to the common resource 806 and the assay bays 804A, 804B (e.g., via a gantry). Moreover, the common resource mover 807 may be movable in the first direction (e.g., parallel to the assay bays) and a second direction perpendicular to the first direction, allowing the mover 807 to traverse the four zones of each assay bay 804A, 804B (eight zones in total) during a workflow. In at least some aspects, the spatial zone may extend from the surface of the assay bays 804A, 804B upward, as the mover 807 may be moving above one or more of the assay bays 804A, 804B (e.g., via the gantry), and may still cause a conflict in a spatial zone even when moving above the surface. For example, the mover 807 may be carrying a liquid reagent while traveling above an assay bay 804B and risk contaminating the assay bay 804B with the reagent while overhead.

### Spatial Resource Collision Detection

In at least some embodiments, the scheduler or other component of the systems 100, 200 may identify a spatial conflict of the movable common resource component (e.g., mover 807) of the common resource 806 and a movable assay bay component (e.g., contact dispensers 805A, 805B) of one or more assay bays 804A, 804B. In at least some aspects, the spatial conflict may include one or more tasks of different workflows which occur in a same spatial zone during the same time. For example, a conflict may occur where a contact dispenser 805A, 805B and mover 807 are scheduled to perform operations, such as a steps of a diluting process involving both movable components, at the same time and in the same spatial zone. In at least some aspects, the spatial conflict may be due to a potential collision according to trajectories of the movable common resource component (e.g., mover 807) and the movable assay bay component (e.g., contact dispenser 805A, 805B) with respect to particular spatial zones. For example, to arrive at the consumables area 810, the mover 807 is scheduled to move through a spatial zone 3A in which the contact dispenser 805A is performing a cleanup process. The scheduler may resolve the spatial conflicts, e.g., based upon workflow characteristics. These characteristics may include spatial characteristics, such as a location of a target object (e.g., a consumable, a library sample, a plate, etc.), the location of the movable assay bay component (e.g., a contact dispenser), the location of the movable common resource component (e.g., a mover), or any other suitable workflow characteristic.

In at least some aspects, in response to identifying a spatial conflict, the system 100, 200 may take one or more actions with respect to scheduling the workflow on the common resource schedule to resolve the conflict. These actions may include pausing one or more workflows associated with the conflict which may include pausing the movable components of the paused workflow. Additionally, these actions may include adding a wait time to one or more workflows associated with the conflict such that the movable components of the workflow with the added wait time will no longer spatially conflict with the movable component of another workflow. Moreover, these actions may include causing the movable common resource component or the movable assay bay component to move in a manner which resolves the spatial conflict. For example, the system 100, 200 may change the route for a mover so that the mover arrives at its intended destination using a route which does not conflict with a contact dispenser.

Returning to the example spatial zone block diagram of FIG. 8A, a first workflow may require the first assay bay contact dispenser 805A to perform an operation in spatial zone 2, such as hydrating a dried reagent. A second workflow may require the mover 807 to retrieve a plate 811 from the consumables area 810. The workflow requests for the first and second workflows may indicate that, if scheduled to the common resource schedule by the scheduler at the times requested by each workflow, a spatial conflict would occur in spatial zone 2 due to the contact dispenser 805A operating in spatial zone 2 during a timeframe in which the mover 807 would be moving through spatial zone 2 to reach the plate 811 in the consumables area 810.

To resolve the conflict, the scheduler may evaluate the workflow characteristics of the first and/or second workflows causing the conflict. In one example, the rehydrating step of the first workflow may have workflow characteristics indicating the workflow is medium priority and has a maximum run time of 20 seconds and maximum wait time of 12 seconds. The second workflow may have workflow characteristics indicating it is medium priority and has a minimum wait time of 30 seconds and maximum run time of 8 seconds. To resolve the conflict, the scheduler may apply a rule which adds a wait time of 10 seconds to the first workflow (within the 12 second maximum wait time), allowing the mover 807 to utilize spatial zone 2 for the entirety of 8 second run time to complete its workflow. The first workflow may then complete its workflow by having the contact dispenser 805A utilize spatial zone 2, as the mover 807 will no longer be occupying the spatial zone.

To resolve the spatial conflict using another rule, the scheduler may create a common resource schedule where the second workflow is paused for 20 seconds (within the 30 second minimum wait time) while the first workflow is conducted. In this scenario, the first workflow has a maximum run time of 20 seconds and will be completed by the time the second workflow resumes after the pause, thereby resolving the spatial conflict.

To resolve the spatial conflict using another rule, the scheduler may create a schedule where the mover 807 moves across spatial zone 1 rather than spatial zone 2 to reach the consumables area 810, and then moves the mover 807 downward to a position parallel to spatial zone 2 but within the consumables area 810 to reach plate 811, so the trajectory of the mover 807 no longer includes moving across spatial zone 2 where the contact dispenser 805A is operating. The scheduler may consider applying other rules to resolve the conflict in light of the workflow characteristics of the first and second workflows.

### Parallel Operation

In at least some aspects, the scheduler or other component of the system 100, 200 (e.g., processor 224), may determine that at least two operations of the same workflow on the common resource schedule can be conducted in parallel. Based upon the determination, the scheduler may assign the two operations of the same workflow to the common resource schedule to be executed in parallel. For example, a workflow for a first assay bay may be configured to perform thermocycling and mixing in parallel/at the same time and when resources for both the task tasks are available. Further, if one task includes a shorter run time than the other task (e.g., mixing has a 5 second run time and thermocycling a 10 second run time), there may be flexibility in when the shorter task (e.g., mixing) may start. The ability to run tasks in parallel may avoid conflicts for the common resource without adding run time, providing more efficient workflows.

Concurrently referring to the spatial zones of block diagram of FIG. 8A and the example block diagram of FIG. 8B illustrating scheduled parallel workflows, the first assay bay 804A is scheduled to carry out two tasks simultaneously: (i) task 1 which requires the contact dispenser 805A to utilize spatial zones 1A and 2A. As task 1 does not require the mover 807 or resources of the common resource 806, task 1 is not scheduled to the common resource schedule; and (ii) task 2 which requires the mover 807 to utilize spatial zones 3A and 4A, and thus is scheduled on the common resource schedule. To get to spatial zones 3A and 3B, the trajectory of the mover 807 will include crossing through spatial zone 4B to arrive at spatial zone 4A. While the second assay bay 804B is not directly involved in task 2, it nonetheless includes a reservation on the common resource schedule to allow the mover 807 to move through spatial zone 4B on its way to spatial zone 4A. Although task 2 is scheduled to start at time t2 on the common resource schedule, task 2 only utilizes spatial zones 3A, 4A and 4B and does not utilize spatial zone 1A, 2A. Therefore, as soon as all of spatial zones 3A, 4A and 4B are available, task 2 may be initiated. Accordingly, the common resource 806 (e.g., the mover 807) may have a conflict just before time t1 with task 0 occurring in spatial zone 3A. However, anytime after time t1, the common resource 806 does not have a conflict and the scheduler can schedule task 2 to start at a time after time t1 to resolve the conflict. In this example, the scheduler schedules task 2 to start at time t2.

Thus, not only has the scheduler optimized the workflows by scheduling them to the common resource schedule to run in parallel rather than sequentially, but task 2 may also be moved up on the common resource schedule to execute at time t1 rather than time t2, using even less time to complete both tasks 1 and task 2.

While the previously described embodiments, aspects, and/or examples may include a first and second assay bay, one common resource and one consumables area, one having skill in the art will understand that the techniques applied to schedule workflows to a common resource schedule in a manner which avoids conflicts may be applied to other configurations of the systems which may include different quantities of assay bays, common resources and/or consumables areas. One having skill in the art will further appreciate that while the rules applies by the scheduler are generally described and/or applied one-by-one, any number and/or combination of rules may be applied to resolve workflow conflicts.

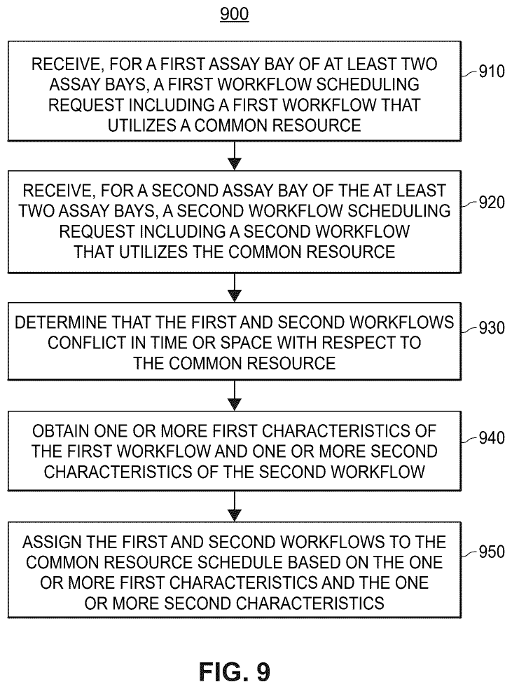

FIG. 9 depicts a flow diagram of an example computer-implemented method 900 for managing multiple parallel library preparation workflows with shared resources. The computer-implemented method 900 may operate as a stand-alone method, may operate in conjunction with other systems and/or methods described herein, and/or may operate in conjunction with embodiments of at least a portion of the system 100 of FIG. 1, system 200 of FIGS. 2-4, and/or of any one or more components and/or devices related thereto, and/or with other systems, processors, databases and/or devices. For example, the scheduler 225 and/or controller 176 may execute at least a portion of the computer-implemented method 900. The computer-implemented method 900 may include additional or alternate steps other than those described with respect to FIG. 9, in embodiments.

Generally speaking, the computer-implemented method 900 may manage multiple parallel library preparation workflows with shared resources, such as common resource 306, 306A, 506, 606, 806. The computer-implemented method 900 may include receiving, by one or more processors, for a first assay bay of at least two assay bays, a first workflow scheduling request including a first workflow that utilizes a common resource (block 910). Each assay bay of the at least two assay bays may be configured to prepare a library of samples according to a workflow including workflow characteristics. The common resource may be configured to assist in preparation of a library of samples according to a common resource schedule.

The computer-implemented method 900 may include receiving, by one or more processors, for a second assay bay of the at least two assay bays, a second workflow scheduling request including a second workflow that utilizes the common resource (block 920).

The computer-implemented method 900 may include determining, by the one or more processors, that the first and second workflows conflict in time or space with respect to the common resource (block 930).

The computer-implemented method 900 may include obtaining, by the one or more processors, one or more first characteristics of the first workflow and one or more second characteristics of the second workflow (block 940). The one or more first and second characteristics include temporal characteristics may include one or more of: a maximum wait time, a minimum wait time, a maximum run time, a minimum run time, a proposed scheduled time, or a priority level.

The computer-implemented method 900 may include assigning, by the one or more processors, the first and second workflows to the common resource schedule based on the one or more first characteristics and the one or more second characteristics (block 950), wherein the common resource schedule resolves the conflict between the first and second workflows with respect to the common resource.

In at least some aspects of the computer-implemented method 900, a first portion of the first workflow and a second portion of the second workflow that utilize the common resource may be assigned to the common resource schedule in an order in which the respective first and second workflow requests are received by the scheduler. Assigning the first and second portions of the first and second workflow to the common resource schedule in the order in which the respective first and second workflow requests are received may further include determining, by the one or more processors, that the first workflow request is received before the second workflow request; and adding, by the one or more processors, a wait time to a requested time of the second portion until the common resource becomes available.

In at least some aspects of the computer-implemented method 900, the one or more first characteristics include a first priority level for the first workflow, the one or more second characteristics include a second priority level for the second workflow, and assigning the first and second workflows to the common resource schedule based on the one or more first characteristics and the one or more second characteristics may further include assigning, by the one or more processors, a first portion of the first workflow and a second portion of the second workflow that requires use of the common resource to the common resource schedule in an order according to the first and second priority levels of the first and second workflows, wherein a higher priority workflow is scheduled before a lower priority workflow.

In at least some aspects, the computer-implemented method 900 may include assigning, by the one or more processors, a second portion of the second workflow that utilizes the common resource to the common resource schedule at an intermediate time between a start of a first portion of the first workflow that utilizes the common resource and an end of the first portion. The one or more first characteristics may include a maximum wait time of the first workflow, and assigning the second portion to the common resource schedule at the intermediate time between the start and end of the first portion may include verifying, by the one or more processors, that a duration of the second portion does not exceed the maximum wait time of the first workflow.

In at least some aspects, the computer-implemented method 900 may include receiving, by the one or more processors, a third workflow scheduling request for a future workflow that utilizes the common resource, wherein the third workflow scheduling request includes a proposed time for the future workflow; determining, by the one or more processors, that the common resource is available at the proposed time; and assigning, by the one or more processors, the future workflow to the common resource schedule at the proposed time.

In at least some aspects, the computer-implemented method 900 may include identifying, by the one or more processors, a spatial conflict between a movable common resource component of the common resource and a movable assay bay component of one of the each assay bays based upon the common bay schedule; based upon the spatial conflict, performing, by the one or more processors, one or more of: pausing the first or second workflow associated with the conflict; adding a wait time to the first or second workflow associated with the conflict; or causing the movable common resource component or the movable assay bay component to move in a manner which resolves the spatial conflict. In at least some aspects of the computer-implemented method 900, the one or more first and second characteristics may include spatial characteristics and may include a location of one or more of: a target object, the movable assay bay component, or the movable common resource component.

In at least some aspects, the computer-implemented method 900 may include identifying, by the one or more processors, spatial zones representing spaces in which the movable common resource component or the movable assay bay component moves during the first or second workflow, wherein the spatial conflict between the movable common resource component and the movable assay bay component may be identified based upon at least one of: (i) a plurality of tasks within the first or second workflows occurring in a same spatial zone, or (ii) a potential collision according to trajectories of the movable common resource component and the movable assay bay component to particular spatial zones.

In at least some aspects of the computer-implemented method 900, the movable assay bay component is a contact dispenser controllable via the one or more processors and linearly movable in a first direction parallel to one of the assay bays; and the movable common resource component is a mover controllable via the one or more processors and is operatively coupled to the common resource and the assay bays, and movable in the first direction and a second direction perpendicular to the first direction. The mover may be movable along a gantry system.

In at least some aspects of the computer-implemented method 900, the one or more processors may cause one or more of the movable common resource component or the movable assay bay component to execute at least a portion of the first workflow or the second workflow according to the common resource schedule.

In at least some aspects of the computer-implemented method 900, the one or more first characteristics may include information identifying an assay bay that initiated the first workflow scheduling request, and the computer-implemented method 900 may include pre-allocating, by the one or more processors, blocks of time on the common resource schedule to each of the assay bays for utilizing the common resource; and assigning, by the one or more processors, a first portion of the first workflow that utilizes the common resource to the common resource schedule at a next pre-allocated block of time for the assay bay that initiated the first workflow scheduling request based upon receiving the first workflow request.

In at least some aspects, the computer-implemented method 900 may include determining, by the one or more processors, that at least two operations of the first or second workflows on the common resource schedule can be conducted in parallel; and assigning, by the one or more processors, the at least two operations of the first or second workflows to the common resource schedule to be executed in parallel.

Example 1. A system for managing multiple parallel library preparation workflows with shared resources, the system comprising: one or more processors; a non-transitory computer-readable memory; at least two assay bays, each assay bay configured to prepare a library of samples according to a workflow including workflow characteristics; a common resource configured to assist in preparation of the library of samples according to a common resource schedule; and a scheduler stored on the memory for scheduling workflows according to the common resource schedule that, when executed by the one or more processors, causes the system to: receive, for a first assay bay of the at least two assay bays, a first workflow scheduling request including a first workflow that utilizes the common resource; receive, for a second assay bay of the at least two assay bays, a second workflow scheduling request including a second workflow that utilizes the common resource; determine that the first and second workflows conflict in time or space with respect to the common resource; obtain one or more first characteristics of the first workflow and one or more second characteristics of the second workflow; and assign the first and second workflows to the common resource schedule based on the one or more first characteristics and the one or more second characteristics, wherein the common resource schedule resolves the conflict between the first and second workflows with respect to the common resource.

Example 2. The system of example 1, wherein the one or more first and second characteristics include temporal characteristics including one or more of: a maximum wait time, a minimum wait time, a maximum run time, a minimum run time, a proposed scheduled time, or a priority level.

Example 3. The system of example 1 or 2, wherein a first portion of the first workflow and a second portion of the second workflow that utilize the common resource are assigned to the common resource schedule in an order in which the respective first and second workflow requests are received by the scheduler.

Example 4. The system of example 3 wherein to assign the first and second portions of the first and second workflow to the common resource schedule in the order in which the respective first and second workflow requests are received, the scheduler causes the system to: determine that the first workflow request is received before the second workflow request; and add a wait time to a requested time of the second portion until the common resource becomes available.

Example 5. The system of any one of the preceding examples, wherein the one or more first characteristics include a first priority level for the first workflow, the one or more second characteristics include a second priority level for the second workflow, and wherein to assign the first and second workflows to the common resource schedule based on the one or more first characteristics and the one or more second characteristics, the scheduler causes the system to: assign a first portion of the first workflow and a second portion of the second workflow that requires use of the common resource to the common resource schedule in an order according to the first and second priority levels of the first and second workflows, wherein a higher priority workflow is scheduled before a lower priority workflow.

Example 6. The system of any one of the preceding examples, wherein the scheduler further causes the system to assign a second portion of the second workflow that utilizes the common resource to the common resource schedule at an intermediate time between a start of a first portion of the first workflow that utilizes the common resource and an end of the first portion.

Example 7. The system of example 6, wherein the one or more first characteristics include a maximum wait time of the first workflow, and wherein to assign the second portion to the common resource schedule at the intermediate time between the start and end of the first portion, the scheduler causes the system to verify that a duration of the second portion does not exceed the maximum wait time of the first workflow.

Example 8. The system of any one of the preceding examples, wherein the scheduler further causes the system to: receive a third workflow scheduling request for a future workflow that utilizes the common resource, wherein the third workflow scheduling request includes a proposed time for the future workflow; determine that the common resource is available at the proposed time; and assign the future workflow to the common resource schedule at the proposed time.

Example 9. The system of any one of the preceding examples, wherein the scheduler further causes the system to: identify a spatial conflict between a movable common resource component of the common resource and a movable assay bay component of one of the each assay bays based upon the common resource schedule; in response to the spatial conflict, perform one or more of: pause the first or second workflow associated with the conflict; add a wait time to the first or second workflow associated with the conflict; or cause the movable common resource component or the movable assay bay component to move in a manner which resolves the spatial conflict

Example 10. The system of example 9, wherein the scheduler further causes the system to: identify spatial zones representing spaces in which the movable common resource component or the movable assay bay component moves during the first or second workflow, wherein the spatial conflict between the movable common resource component and the movable assay bay component is identified based upon at least one of: (i) a plurality of tasks within the first or second workflows occurring in a same spatial zone, or (ii) a potential collision according to trajectories of the movable common resource component and the movable assay bay component to particular spatial zones.

Example 11. The system of example 9, wherein: the movable assay bay component is a contact dispenser controllable via the one or more processors and linearly movable in a first direction parallel to one of the assay bays, and the movable common resource component is a mover controllable via the one or more processors and is operatively coupled to the common resource and the assay bays, and movable in the first direction and a second direction perpendicular to the first direction.

Example 12. The system example 11, wherein the mover is movable along a gantry system.

Example 13. The system of example 9, wherein the one or more first and second characteristics include spatial characteristics and include a location of one or more of: a target object, the movable assay bay component, or the movable common resource component.

Example 14. The system of any example 9, wherein the one or more processors cause one or more of the movable common resource component or the movable assay bay component to execute at least a portion of the first workflow or the second workflow according to the common resource schedule.

Example 15. The system of any one of examples 1-14, wherein the one or more first characteristics include information identifying an assay bay that initiated the first workflow scheduling request, and wherein the scheduler further causes the system to: pre-allocate blocks of time on the common resource schedule to each of the assay bays for utilizing the common resource; and in response to receiving the first workflow request, assign a first portion of the first workflow that utilizes the common resource to the common resource schedule at a next pre-allocated block of time for the assay bay that initiated the first workflow scheduling request.

Example 16. The system of any one of examples 1-15, wherein the scheduler further causes the system to: determine that at least two operations of the first or second workflows on the common resource schedule can be conducted in parallel; and assign the at least two operations of the first or second workflows to the common resource schedule to be executed in parallel.

Example 17. A computer-implemented method for managing multiple parallel library preparation workflows with shared resources, the method comprising: receiving, by one or more processors, for a first assay bay of at least two assay bays, a first workflow scheduling request including a first workflow that utilizes a common resource, wherein each assay bay of the at least two assay bays is configured to prepare a library of samples according to a workflow including workflow characteristics; and the common resource is configured to assist in preparation of a library of samples according to a common resource schedule; receiving, by one or more processors, for a second assay bay of the at least two assay bays, a second workflow scheduling request including a second workflow that utilizes the common resource; determining, by the one or more processors, that the first and second workflows conflict in time or space with respect to the common resource; obtaining, by the one or more processors, one or more first characteristics of the first workflow and one or more second characteristics of the second workflow; and assigning, by the one or more processors, the first and second workflows to the common resource schedule based on the one or more first characteristics and the one or more second characteristics, wherein the common resource schedule resolves the conflict between the first and second workflows with respect to the common resource.

Example 18. The computer-implemented method of example 17, wherein a first portion of the first workflow and a second portion of the second workflow that utilize the common resource are assigned to the common resource schedule in an order in which the respective first and second workflow requests are received by the scheduler.

Example 19. The computer-implemented method of example 18, wherein assigning the first and second portions of the first and second workflow to the common resource schedule in the order in which the respective first and second workflow requests are received, further comprises: determining, by the one or more processors, that the first workflow request is received before the second workflow request; and adding, by the one or more processors, a wait time to a requested time of the second portion until the common resource becomes available.

Example 20. The computer-implemented method of any one of examples 17-19, wherein the one or more first characteristics include a first priority level for the first workflow, the one or more second characteristics include a second priority level for the second workflow, and wherein assigning the first and second workflows to the common resource schedule based on the one or more first characteristics and the one or more second characteristics, further comprises: assigning, by the one or more processors, a first portion of the first workflow and a second portion of the second workflow that requires use of the common resource to the common resource schedule in an order according to the first and second priority levels of the first and second workflows, wherein a higher priority workflow is scheduled before a lower priority workflow.

Example 21. The computer-implemented method of any one of examples 17-20, further comprising assigning, by the one or more processors, a second portion of the second workflow that utilizes the common resource to the common resource schedule at an intermediate time between a start of a first portion of the first workflow that utilizes the common resource and an end of the first portion.

Example 22. The computer-implemented method of example 21, wherein the one or more first characteristics include a maximum wait time of the first workflow, and wherein assigning the second portion to the common resource schedule at the intermediate time between the start and end of the first portion, further comprises verifying, by the one or more processors, that a duration of the second portion does not exceed the maximum wait time of the first workflow.

Example 23. The computer-implemented method of any one of examples 17-22, further comprising: receiving, by the one or more processors, a third workflow scheduling request for a future workflow that utilizes the common resource, wherein the third workflow scheduling request includes a proposed time for the future workflow; determining, by the one or more processors, that the common resource is available at the proposed time; and assigning, by the one or more processors, the future workflow to the common resource schedule at the proposed time.

Example 24. The computer-implemented method of any one of examples 17-23, further comprising: identifying, by the one or more processors, a spatial conflict between a movable common resource component of the common resource and a movable assay bay component of one of the each assay bays based upon the common resource schedule; based upon the spatial conflict, performing, by the one or more processors, one or more of: pausing the first or second workflow associated with the conflict; adding a wait time to the first or second workflow associated with the conflict; or causing the movable common resource component or the movable assay bay component to move in a manner which resolves the spatial conflict.

Example 25. The computer-implemented method of example 24, further comprising: identifying, by the one or more processors, spatial zones representing spaces in which the movable common resource component or the movable assay bay component moves during the first or second workflow, wherein the spatial conflict between the movable common resource component and the movable assay bay component is identified based upon at least one of: (i) a plurality of tasks within the first or second workflows occurring in a same spatial zone, or (ii) a potential collision according to trajectories of the movable common resource component and the movable assay bay component to particular spatial zones.

Example 26. The computer-implemented method of example 25, further comprising: causing, by the one or more processors, one or more of the movable common resource component or the movable assay bay component to execute at least a portion of the first workflow or the second workflow according to the common resource schedule.

Example 27. The computer-implemented method of any one of examples 17-26, wherein the one or more first characteristics include information identifying an assay bay that initiated the first workflow scheduling request, and further comprising: pre-allocating, by the one or more processors, blocks of time on the common resource schedule to each of the assay bays for utilizing the common resource; and assigning, by the one or more processors, a first portion of the first workflow that utilizes the common resource to the common resource schedule at a next pre-allocated block of time for the assay bay that initiated the first workflow scheduling request based upon receiving the first workflow request.

Example 28. The computer-implemented method of any one of examples 17-27, further comprising: determining, by the one or more processors, that at least two operations of the first or second workflows on the common resource schedule can be conducted in parallel; and assigning, by the one or more processors, the at least two operations of the first or second workflows to the common resource schedule to be executed in parallel.

Example 29. A non-transitory computer-readable medium storing processor-executable instructions that, when executed by one or more processors, cause the one or more processors to: receive, for a first assay bay of at least two assay bays, a first workflow scheduling request including a first workflow that utilizes a common resource, wherein each assay bay of the at least two assay bays is configured to prepare a library of samples according to a workflow including workflow characteristics; and the common resource is configured to assist in preparation of a library of samples according to a common resource schedule; receive, for a second assay bay of the at least two assay bays, a second workflow scheduling request including a second workflow that utilizes the common resource; determine that the first and second workflows conflict in time or space with respect to the common resource; obtain one or more first characteristics of the first workflow and one or more second characteristics of the second workflow; and assign the first and second workflows to the common resource schedule based on the one or more first characteristics and the one or more second characteristics, wherein the common resource schedule resolves the conflict between the first and second workflows with respect to the common resource.

Example 30. The non-transitory computer-readable medium of example 29, wherein a first portion of the first workflow and a second portion of the second workflow that utilize the common resource are assigned to the common resource schedule in an order in which the respective first and second workflow requests are received by the scheduler.

Example 31. The non-transitory computer-readable medium of example 30, wherein to assign the first and second portions of the first and second workflow to the common resource schedule in the order in which the respective first and second workflow requests are received, the instructions further cause the one or more processors to: determine that the first workflow request is received before the second workflow request; and add a wait time to a requested time of the second portion until the common resource becomes available.

Example 32. The non-transitory computer-readable medium of examples 29 to 31, wherein the one or more first characteristics include a first priority level for the first workflow, the one or more second characteristics include a second priority level for the second workflow, and wherein to assign the first and second workflows to the common resource schedule based on the one or more first characteristics and the one or more second characteristics, the instructions further cause the one or more processors to: assign a first portion of the first workflow and a second portion of the second workflow that requires use of the common resource to the common resource schedule in an order according to the first and second priority levels of the first and second workflows, wherein a higher priority workflow is scheduled before a lower priority workflow.

Example 33. The non-transitory computer-readable medium of examples 29 to 32, wherein the instructions further cause the one or more processors to assign a second portion of the second workflow that utilizes the common resource to the common resource schedule at an intermediate time between a start of a first portion of the first workflow that utilizes the common resource and an end of the first portion.

Example 34. The non-transitory computer-readable medium of example 33, wherein the one or more first characteristics include a maximum wait time of the first workflow, and wherein to assign the second portion to the common resource schedule at the intermediate time between the start and end of the first portion, the instructions further cause the one or more processors to verify that a duration of the second portion does not exceed the maximum wait time of the first workflow.

Example 35. The non-transitory computer-readable medium of examples 29 to 34, wherein the instructions further cause the one or more processors to: receive a third workflow scheduling request for a future workflow that utilizes the common resource, wherein the third workflow scheduling request includes a proposed time for the future workflow; determine that the common resource is available at the proposed time; and assign the future workflow to the common resource schedule at the proposed time.

Example 36. The non-transitory computer-readable medium of examples 29 to 35, wherein the instructions further cause the one or more processors to: identify a spatial conflict between a movable common resource component of the common resource and a movable assay bay component of one of the each assay bays based upon the common resource schedule; in response to the spatial conflict, perform one or more of: pause the first or second workflow associated with the conflict; add a wait time to the first or second workflow associated with the conflict; or cause the movable common resource component or the movable assay bay component to move in a manner which resolves the spatial conflict.

Example 37. The non-transitory computer-readable medium of example 36, wherein the instructions further cause the one or more processors to: identify spatial zones representing spaces in which the movable common resource component or the movable assay bay component moves during the first or second workflow, wherein the spatial conflict between the movable common resource component and the movable assay bay component is identified based upon at least one of: (i) a plurality of tasks within the first or second workflows occurring in a same spatial zone, or (ii) a potential collision according to trajectories of the movable common resource component and the movable assay bay component to particular spatial zones.

Example 38. The non-transitory computer-readable medium of example 36, wherein the instructions further cause the one or more processors to cause one or more of the movable common resource component or the movable assay bay component to execute at least a portion of the first workflow or the second workflow according to the common resource schedule.

Example 39. The non-transitory computer-readable medium of examples 29 to 38, wherein the one or more first characteristics include information identifying an assay bay that initiated the first workflow scheduling request, and the instructions further cause the one or more processors to: pre-allocate blocks of time on the common resource schedule to each of the assay bays for utilizing the common resource; and in response to receiving the first workflow request, assign a first portion of the first workflow that utilizes the common resource to the common resource schedule at a next pre-allocated block of time for the assay bay that initiated the first workflow scheduling request.

Example 40. The non-transitory computer-readable medium of examples 29 to 39, wherein the instructions further cause the one or more processors to: determine that at least two operations of the first or second workflows on the common resource schedule can be conducted in parallel; and assign the at least two operations of the first or second workflows to the common resource schedule to be executed in parallel.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

As used herein, an element or step recited in the singular and proceeded with the word “a” or “an” should be understood as not excluding plural of said elements or steps, unless such exclusion is explicitly stated. Furthermore, references to “one implementation” are not intended to be interpreted as excluding the existence of additional implementations that also incorporate the recited features. Moreover, unless explicitly stated to the contrary, implementations “comprising,” “including,” or “having” an element or a plurality of elements having a particular property may include additional elements whether or not they have that property. Moreover, the terms “comprising,” including,” having,” or the like are interchangeably used herein.

The terms “substantially,” “approximately,” and “about” used throughout this Specification are used to describe and account for small fluctuations, such as due to variations in processing. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%. In one example, these terms include situation where there is no variation—0%.

There may be many other ways to implement the subject technology. Various functions and elements described herein may be partitioned differently from those shown without departing from the scope of the subject technology. Various modifications to these implementations may be readily apparent to those skilled in the art, and generic principles defined herein may be applied to other implementations. Thus, many changes and modifications may be made to the subject technology, by one having ordinary skill in the art, without departing from the scope of the subject technology. For instance, different numbers of a given module or unit may be employed, a different type or types of a given module or unit may be employed, a given module or unit may be added, or a given module or unit may be omitted.

Underlined and/or italicized headings and subheadings are used for convenience only, do not limit the subject technology, and are not referred to in connection with the interpretation of the description of the subject technology. All structural and functional equivalents to the elements of the various implementations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

It should be appreciated that all combinations of the foregoing concepts and additional concepts discussed in greater detail below (provided such concepts are not mutually inconsistent) are contemplated as being part of the subject matter disclosed herein. In particular, all combinations of claimed subject matter appearing at the end of this disclosure are contemplated as being part of the subject matter disclosed herein.

What is claimed is:

1. A system for managing multiple parallel library preparation workflows with shared resources, the system comprising:
one or more processors;
a non-transitory computer-readable memory;
at least two assay bays, each assay bay configured to prepare a library of samples according to a workflow including workflow characteristics;
a common resource configured to assist in preparation of the library of samples according to a common resource schedule; and
a scheduler stored on the memory for scheduling workflows according to the common resource schedule that, when executed by the one or more processors, causes the system to:
receive, for a first assay bay of the at least two assay bays, a first workflow scheduling request including a first workflow that utilizes the common resource;
receive, for a second assay bay of the at least two assay bays, a second workflow scheduling request including a second workflow that utilizes the common resource;
determine that the first and second workflows conflict in time or space with respect to the common resource;
obtain one or more first characteristics of the first workflow and one or more second characteristics of the second workflow; and
assign the first and second workflows to the common resource schedule based on the one or more first characteristics and the one or more second characteristics, wherein the common resource schedule resolves the conflict between the first and second workflows with respect to the common resource.

2. The system of claim 1, wherein the one or more first and second characteristics include temporal characteristics including one or more of: a maximum wait time, a minimum wait time, a maximum run time, a minimum run time, a proposed scheduled time, or a priority level.

3. The system of claim 1, wherein a first portion of the first workflow and a second portion of the second workflow that utilize the common resource are assigned to the common resource schedule in an order in which the respective first and second workflow requests are received by the scheduler.

4. The system of claim 3, wherein to assign the first and second portions of the first and second workflow to the common resource schedule in the order in which the respective first and second workflow requests are received, the scheduler causes the system to:
determine that the first workflow request is received before the second workflow request; and
add a wait time to a requested time of the second portion until the common resource becomes available.

5. The system of claim 1, wherein the one or more first characteristics include a first priority level for the first workflow, the one or more second characteristics include a second priority level for the second workflow, and wherein to assign the first and second workflows to the common resource schedule based on the one or more first characteristics and the one or more second characteristics, the scheduler causes the system to:
assign a first portion of the first workflow and a second portion of the second workflow that requires use of the common resource to the common resource schedule in an order according to the first and second priority levels of the first and second workflows, wherein a higher priority workflow is scheduled before a lower priority workflow.

6. The system of claim 1, wherein the scheduler further causes the system to assign a second portion of the second workflow that utilizes the common resource to the common resource schedule at an intermediate time between a start of a first portion of the first workflow that utilizes the common resource and an end of the first portion.

7. The system of claim 6, wherein the one or more first characteristics include a maximum wait time of the first workflow, and wherein to assign the second portion to the common resource schedule at the intermediate time between the start and end of the first portion, the scheduler causes the system to verify that a duration of the second portion does not exceed the maximum wait time of the first workflow.

8. The system of claim 1, wherein the scheduler further causes the system to:
receive a third workflow scheduling request for a future workflow that utilizes the common resource, wherein the third workflow scheduling request includes a proposed time for the future workflow;
determine that the common resource is available at the proposed time; and
assign the future workflow to the common resource schedule at the proposed time.

9. The system of claim 1, wherein the scheduler further causes the system to:
identify a spatial conflict between a movable common resource component of the common resource and a movable assay bay component of one of the each assay bays based upon the common resource schedule;
in response to the spatial conflict, perform one or more of:
pause the first or second workflow associated with the conflict;
add a wait time to the first or second workflow associated with the conflict; or
cause the movable common resource component or the movable assay bay component to move in a manner which resolves the spatial conflict.

10. The system of claim 9, wherein the scheduler further causes the system to:
identify spatial zones representing spaces in which the movable common resource component or the movable assay bay component moves during the first or second workflow, wherein the spatial conflict between the movable common resource component and the movable assay bay component is identified based upon at least one of:
(i) a plurality of tasks within the first or second workflows occurring in a same spatial zone, or
(ii) a potential collision according to trajectories of the movable common resource component and the movable assay bay component to particular spatial zones.

11. The system of claim 9, wherein:
the movable assay bay component is a contact dispenser controllable via the one or more processors and linearly movable in a first direction parallel to one of the assay bays, and
the movable common resource component is a mover controllable via the one or more processors and is operatively coupled to the common resource and the assay bays, and movable in the first direction and a second direction perpendicular to the first direction.

12. The system of claim 11, wherein the mover is movable along a gantry system.

13. The system of claim 9, wherein the one or more first and second characteristics include spatial characteristics and include a location of one or more of: a target object, the movable assay bay component, or the movable common resource component.

14. The system of claim 9, wherein the one or more processors cause one or more of the movable common resource component or the movable assay bay component to execute at least a portion of the first workflow or the second workflow according to the common resource schedule.

15. The system of claim 1, wherein the one or more first characteristics include information identifying an assay bay that initiated the first workflow scheduling request, and wherein the scheduler further causes the system to:
pre-allocate blocks of time on the common resource schedule to each of the assay bays for utilizing the common resource; and
in response to receiving the first workflow request, assign a first portion of the first workflow that utilizes the common resource to the common resource schedule at a next pre-allocated block of time for the assay bay that initiated the first workflow scheduling request.

16. The system of claim 1, wherein the scheduler further causes the system to:
determine that at least two operations of the first or second workflows on the common resource schedule can be conducted in parallel; and
assign the at least two operations of the first or second workflows to the common resource schedule to be executed in parallel.

17. A computer-implemented method for managing multiple parallel library preparation workflows with shared resources, the method comprising:
receiving, by one or more processors, for a first assay bay of at least two assay bays, a first workflow scheduling request including a first workflow that utilizes a common resource, wherein
each assay bay of the at least two assay bays is configured to prepare a library of samples according to a workflow including workflow characteristics; and
the common resource is configured to assist in preparation of a library of samples according to a common resource schedule;
receiving, by one or more processors, for a second assay bay of the at least two assay bays, a second workflow scheduling request including a second workflow that utilizes the common resource;
determining, by the one or more processors, that the first and second workflows conflict in time or space with respect to the common resource;
obtaining, by the one or more processors, one or more first characteristics of the first workflow and one or more second characteristics of the second workflow; and
assigning, by the one or more processors, the first and second workflows to the common resource schedule based on the one or more first characteristics and the one or more second characteristics, wherein the common resource schedule resolves the conflict between the first and second workflows with respect to the common resource.

18. The computer-implemented method of claim 17, wherein a first portion of the first workflow and a second portion of the second workflow that utilize the common resource are assigned to the common resource schedule in an order in which the respective first and second workflow requests are received by the scheduler.

19. The computer-implemented method of claim 18, wherein assigning the first and second portions of the first and second workflow to the common resource schedule in the order in which the respective first and second workflow requests are received, further comprises:
determining, by the one or more processors, that the first workflow request is received before the second workflow request; and
adding, by the one or more processors, a wait time to a requested time of the second portion until the common resource becomes available.

20. A non-transitory computer-readable medium storing processor-executable instructions that, when executed by one or more processors, cause the one or more processors to:
receive, for a first assay bay of at least two assay bays, a first workflow scheduling request including a first workflow that utilizes a common resource, wherein
each assay bay of the at least two assay bays is configured to prepare a library of samples according to a workflow including workflow characteristics; and
the common resource is configured to assist in preparation of a library of samples according to a common resource schedule;
receive, for a second assay bay of the at least two assay bays, a second workflow scheduling request including a second workflow that utilizes the common resource;
determine that the first and second workflows conflict in time or space with respect to the common resource;
obtain one or more first characteristics of the first workflow and one or more second characteristics of the second workflow; and
assign the first and second workflows to the common resource schedule based on the one or more first characteristics and the one or more second characteristics, wherein the common resource schedule resolves the conflict between the first and second workflows with respect to the common resource.
